(19) United States
(12) Patent Application Publication
(10) Pub. No.: US 2014/0234974 A1
(43) Pub. Date: Aug. 21, 2014

(54) USE OF GENE REGULATORY NETWORK LOGIC FOR TRANSFORMATION OF CELLS

(71) Applicant: California Institute of Technology, Pasadena, CA (US)

(72) Inventors: Isabelle Peter, South Pasadena, CA (US); Yong Zhu, Anaheim, CA (US); Eric H. Davidson, Pasadena, CA (US)

(73) Assignee: CALIFORNIA INSTITUTE OF TECHNOLOGY, Pasadena, CA (US)

(21) Appl. No.: 14/181,280

(22) Filed: Feb. 14, 2014

Related U.S. Application Data
(60) Provisional application No. 61/765,451, filed on Feb. 15, 2013.

(51) Int. Cl. G06F 19/12; C12N 5/071; C12N 5/0783; C12N 5/077

(52) U.S. Cl. CPC G06F 19/12; C12N 5/0655; C12N 5/0676; C12N 5/0637
USPC 435/467; 435/377; 435/455; 703/11

(57) ABSTRACT

Described herein is a gene regulatory network based focused approach to cell transformation. The methods described herein allow for identification of circuit and sub-circuit repertoires for which modification in a starting cell type can result in generation of a transformed cell type in a durable and persistent manner, without requiring potentially deleterious genome modification. The described methods and compositions produced by the methods find widespread application in regenerative medicine applications.

### CROSS-REFERENCE TO OTHER APPLICATIONS

This application includes a claim of priority under 35 U.S.C. §119(e) to U.S. provisional patent applications No. 61/765,451, filed Feb. 15, 2013.

### FIELD OF THE INVENTION

The claimed invention relates to regenerative medicine applications by providing a gene regulatory network (GRN) based approach for transforming cells to generate transplantable cellular materials.

### BACKGROUND

Despite great advances in biomedical research, effective treatments for some of the most devastating diseases, such as diabetes, cardiovascular diseases, spinal cord injury, Parkinson's disease, and Alzheimer's disease may benefit greatly from the development of regenerative medicine technology, such as generation transplantable cellular material, as a chief aspect of these diseases involves damage, depletion or deficiency of certain type of cells. Many recent advances in regenerative medicine have arisen from isolation of human embryonic stem cells (ESCs) capable of differentiating into virtually every type of cells in a human body. While early concepts visualized differentiation from ESCs to progenitor cells and somatic cells as an irreversible process, this notion was debunked following the discovery of induced pluripotent stem cells (iPSCs), wherein somatic cells have been established as capable of reprogramming to pluripotent stem cells following introduction of reprogramming factors, such as Oct4, Klf4, Sox2 and c-Myc. Such results clearly indicate that cell fate is not fixed, that somatic cells can be reversed back to pluripotent stem cells, and also implicating conversion to other type of somatic cells, upon introduction of reprogramming factors. Importantly, iPSC-related discoveries also demonstrate that fibroblast cells can be converted to iPSCs by introducing recombinant proteins. By eliminating the risks associated with target cell genome modifications, which may have deleterious effects, the opportunities for use of stem cells, derivative and related cells for therapeutic purposes is greatly enhanced.

Nevertheless, despite these important advances, studies focusing on controlling the fates of uncommitted cells such as embryonic stem cells, stem cells in mature tissues and other progenitor cells, have only touched the surface of the intricate genetic program and regulatory system involved in the adoption of cell fate decisions. For example, while pluripotent and progenitor stem cells have been studied for their ability to adopt the fade of, and differentiate into, various terminally differentiated lineages, the morphogenic factors and culture conditions applied for these purposes have mainly been achieved by laborious trial and error. Studies focused on differentiation cells towards a certain fate frequently result in mixed populations, providing a high variable readout of indeterminate or unsatisfactory quality as related to cell identity. For example, confirmation of differentiation state is frequently determined through phenotypic observation or monitoring only a selective number of known markers, thereby failing to confirm the true identity of differentiated cellular products, further including potentially undesirable cellular activities. Until more precise characterization of genetic regulatory systems governing differentiation states are established, such cells may be of limited value for harnessing the full diagnostic or therapeutic potential of generating transplantable cellular material.

Accordingly, absent characterization of the genetic regulatory system as a whole for the spatial and temporal complexity of cell fate decision and the resulting differentiation, development, repair, remodeling and renewal processes, the authenticity and reliability of various directed differentiation techniques will be insufficiently complete for confident use in the diagnosis and treatment of diseases. Thus, there exists a need for the identification and characterization of the genetic regulatory architecture of a cell and for developing techniques exploiting these architectures for variable formation of defined cellular states.

Described herein is the application of gene regulatory network (GRN) based approach for characterizing developmental and cellular functions at a systematic level in the terms of genomic regulatory architecture. Depending on their developmental functions, GRNs can differ in their degree of hierarchy, and also in the types of modular circuits and sub-circuits of which they are composed. The techniques described herein allow for identification of circuit and sub-circuit repertoires for which modification in a starting cell type can result in generation of a transformed cell type in a durable and persistent manner, without requiring potentially deleterious genome modification and in a predictable manner defined by the underlying genetic regulatory architecture of a cell.

### BRIEF DESCRIPTION OF THE FIGURES

Exemplary embodiments are illustrated in referenced figures. It is intended that the embodiments and figures disclosed herein are to be considered illustrative rather than restrictive.

The patent or application file contains at least one drawing executed in color. Copies of this patent or patent application publication with color drawing(s) will be provided by the Office upon request and payment of the necessary fee.

FIG. 1. Exemplary Sub-Circuit Repertoires. A variety of well-known sub-circuit repertoires are depicted, including (A) Certain sub-circuits associated with early body planning and embryonic development (B) Dynamic lockdown of the regulatory state (C) Various terminal processes related to regulatory or maintenance processes in the cell. The role of the sub-circuit is given in column 1; its name in column 2; a description of its function in column 3; and the sub-circuit structure in column 4, Numbers in column 2 are keyed to FIG. 1, In Topologies column; all genes encode transcription factors unless otherwise noted. * Regulatory genes that create initial regulatory state are controlled by widely expressed repressor, which is dominant over their positive inputs, and gene encoding this repressor is itself specifically repressed in a local region (X) by another gene encoding a different repressor: hence target genes are ON in X, specifically, repressed elsewhere. † Many developmental signaling systems (for example, Notch, Wnt) activate immediate early response factors in cells receiving ligand, but in absence of ligand, these factors act as dominant repressors of the same target genes. ‡ Dynamic in that continuing transcription is required. § Exclusion sub-circuits are activated as downstream outputs of specification GRNs. ∥ A unique circuit design here is that the ligand gene is activated by the same signal transduction mechanism reception of the ligand activates in recipient cells; a positive intercellular feedback, ¶ Expanded discussion can be found in Peter, I. S. & Davidson, E. H. Modularity and design principles in the sea urchin embryo gene regulatory: network. FEB Lett. 583, 3948-3958 (2009), herein are incorporated by reference in their entirety as though fully set forth. #L, gene encoding signaling ligand. ⋆ R encodes repressor; L encodes signaling ligand, †† Conceived as a means of obtaining different discrete transcriptional responses from a graded signal; see discussion of this type of circuitry in section on mathematical models below, ‡‡ S, signal; triangle represents graded signal strength §§ S1, S2, different signal inputs gene B is subject to additional transcriptional repression in certain regulatory states. ∥∥ This design precludes necessity for ad hoc Hill coefficients as in 5, 6.1; see section on mathematical models below. ¶¶ Autoregulatory loops lock on whichever state the system goes to. (Adapted from Davidson, Eric H. (2010) Emerging properties of animal gene regulatory networks. Nature, 468 (7326). pp. 911-920)

FIG. 2. Structural characteristics of downstream effector gene cassettes and their control functions. (A) Typical differentiation gene battery. Here each effector gene codes for a cell-type-specific protein required to generate the cell-specific output. These effector genes are all transcribed specifically in the given cell type in response to a small number of regulatory factors, which are themselves the output of the controlling specification gene regulatory architecture. Every effector gene of the battery is specifically controlled by these inputs. The immediate drivers of the battery shown cross-regulate. (B) Structure that may be typical of morphogenetic effector gene cassettes. Here the output of the specification GRN is used to control transcription of only a minor fraction of key effector genes, and these in some way trigger or nucleate the process. But many of the proteins required for the function are widely expressed. (Adapted from Davidson, Eric H. Nature, 468. pp. 911-920)

FIG. 3. Gene Regulatory Network of β-cell. (A) High level conception of the sub-circuits depicted in FIG. 1 as applied in the context of β-cell development. (B) Precise organization of the various nodes depicted in a network topology. (Adapted from Davidson, Eric H. Nature, 468. pp. 911-920)

FIG. 4. Three Modules: the Core Circuitry of β Cells. Examples of specific sub-circuit modules from the network topology shown in FIG. 3B. Here, the plurality of nodes are depicted in a network topology organized as a self-perpetuating positive feedback loop. Various subcircuits have specific cellular functions, such as causing various endocrine genes to be active, repressing genes of other cell types, accounting for responses to glucose, and other physiological responses, and ensuring stability of regulatory state.

FIG. 5. Transducible Proteins. To enable the transcription factor (TF) proteins enter the cells, the Inventors initially designed and produced fusion proteins tagged with C-terminal poly-Arginine peptides. To further improve the transduction efficiency, one can fuse the transcription factor proteins with supercharged Green Fluorescent Proteins.

FIG. 6. sGFP-MafA efficiently transduced into 293 cells. In an application of the recombinant fusion protein described above, such proteins can be effectively transduced into cells.

FIG. 7. Transformation of liver cells to insulin secreting cells. (A) Experimental design (B) Immunofluorescent analysis of control mouse liver (C) Triple protein treatment (Pdx1-11R, Ngn3-11R, and MafA-11R) produced insulin positive cells in mouse liver in vivo experiments. (D) One Combination (sGFP-Pdx1, -Nkx2.2, and -Nkx6.1) shifted the gene expression profile of HepG2 cells towards that of islet cells as measured via qRT-PCR. (E) Characterization of gene expression profile in human liver cell line THLE-2 compared to human islet cells, showing high divergence of the expression profile. (F) In human liver cell line, demonstration of cell type transformation driven by selected exogenous transcription factors, InsulinEnhancer-mCherry: red incorporated exogenous transcription factors: green.

FIG. 8. Transformation of non-islet cells of the pancreas to insulin-secreting cells—in vivo. (A) Experimental design (B) Immunofluorescent analysis of control mouse pancreas (C) Triple protein treatment (Pdx1-11R, Ngn3-11R, and MafA-11R) produced insulin positive cells in mouse pancreas

FIG. 9. Transformation of peripheral T cells to Treg cells—in vitro (A) Example 3: (B) Foxp3-11R increased the percentage of CD4+CD25Hi cells in a dose-dependent manner as shown via flow cytometry (FACS) (C) Transformation to Treg cells in vivo: evaluation of Foxp3-11R in arthritis mouse model (D) Amelioration of rheumatoid arthritis in mouse model.

FIG. 10. Transformation of mesenchymal stem cells to chondrocytes. (A) Experimental design. (B) Penetration of sGFP-SOX9 protein into HHF and MSC. Human skin fibroblast cell line, HHF (i. and ii.) or human bone marrow derived mesenchymal stem cells (MSC) (iii. And iv.) were incubated with 10 μg/ml of sGFP or sGFP-SOX9 in DMEM at 37° C. for 1 hour. Cells were washed and viewed under fluorescent microscope. i and iii: SGFP; ii and iv: sGFP-SOX9. (C) sGFP-Sox9 increased collagen type II but decreased collagen type I and type X expression. MSC were cultured with DMEM with addition of buffer only or 10 μg/ml of sGFP-SOX9. At the indicated time point (hours), RNA were extracted and RT-PCR was performed with TagMan probe based analysis assay for collagen (Col) type I, II and X mRNA expression, as relative to GAPDH. (D) sGFP-Sox9 increased aggrecan expression. 10 μg/ml of sGFP-SOX9 was added to MSC culture. After 24 hours, the MSCs were changed back to medium without sGFP-SOX9. Culture was maintained for 14 days. (i. MSC with buffer. ii. MSC with sGFPSOX9 treatment at 3 days. iii. MSC with sGFP-SOX9 treatment at 14 days. Toluidine blue staining) Toluidine blue stains aggrecan which is a major component of proteoglycan in articular cartilage matrix. Note the chondrocyte morphology in ii. and iii. and purple staining indicating these cells containing aggrecan.

### SUMMARY OF THE INVENTION

Described herein is a method of transforming a cell including providing a quantity of at least one cis regulatory network element, and introducing into a starting cell type, the at least one cis regulatory network element, wherein the at least one cis regulatory network element is capable of altering a regulatory sub-circuit in the starting cell type, thereby altering one of more properties of the starting cell type, and generating a transformed cell type. In various embodiments, the cis regulatory network element includes a transcription factor and derivatives thereof. In various embodiments, the cis regulatory network element includes a recombinant protein. In various embodiments, the cis regulatory network element is encoded by a nucleic acid. In various embodiments, the regulatory sub-circuit is a positive feedback loop. In various embodiments, the regulatory sub-circuit includes at least two cis regulatory network elements. In various embodiments, the regulatory sub-circuit includes at least three cis regulatory network elements. In various embodiments, the one or more properties includes transcription factor expression and/or transcription factor binding to a cis regulatory network element. In various embodiments, the one or more properties includes protein expression and/or surface marker expression. In various embodiments, the starting cell type is a hepatocyte. In various embodiments, the starting cell type is a non-insulin secreting islet cell. In various embodiments, the transformed cell type is an insulin secreting islet cell. In various embodiments, the insulin secreting islet cell expresses Pdx, MafA and Ngn3. In various embodiments, the starting cell type is a peripheral T cell. In various embodiments, the transformed cell type is a T_{reg} cell. In various embodiments, the T_{reg} cell expresses Foxp3. In various embodiments, the starting cell type is a mesenchymal stem cell. In various embodiments, the transformed cell type is a chondrocyte. In various embodiments, the chondrocyte expresses Sox9.

Also described herein is a quantity of transformed cells made by the method of method of transforming a cell including providing a quantity of at least one cis regulatory network element, and introducing into a starting cell type, the at least one cis regulatory network element, wherein the at least one cis regulatory network element is capable of altering a regulatory sub-circuit in the starting cell type, thereby altering one of more properties of the starting cell type, and generating a transformed cell type.

Also described herein is method for identifying a regulatory network for transforming a cell, including organizing a plurality of cis regulatory network elements into a network topology of nodes comprising circuits, wherein the circuits comprise at least one sub-circuit, and identifying at least one sub-circuit comprising at least one positive effector node, wherein the at least one positive effector node is capable of generating a transformed cell type when introduced into a staring cell. In various embodiments, the sub-circuit is a positive feedback loop. In various embodiments, the sub-circuit comprises at least two cis regulatory network elements. In various embodiments, the sub-circuit comprises at least three cis regulatory network elements.

Further described herein is a composition including a quantity of cells expressing at least one exogenously added protein, wherein the at least one exogenously added protein is a positive effector in a regulatory sub-circuit. In various embodiments, the cells express at least two exogenously added proteins, and the at least two exogenously added proteins are each positive effectors in a regulatory sub-circuit. In various embodiments, the regulatory sub-circuit is a positive feedback loop.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul., 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods described herein. For purposes of the present invention, the following terms are defined below.

As used herein, the term “cis regulatory network” is intended to mean a collection of transcription and/or signaling factors, cis regulatory nucleic acid sequences, modules, related by binding activity and/or sharing a common function. This can include, for example, transcription factors and their cognate cis regulatory nucleic acid sequences or modules. In a broader sense, the term as used herein can refer to, for example, the total number of connections of cis regulatory modules and transcription or signaling factor elements. In another sense, the term can encompass one or more series of connections, exemplified by circuits and sub-circuits, the various cis regulatory network elements serving as nodes in circuits and sub-circuits, and such with active cis regulatory connections maintaining a relationship of common function. This further includes both temporal and/or spatial order of cis regulatory connections.

As used herein, the term “cis regulatory network interaction” is intended to mean a binding event between network elements of a cis regulatory network. Binding events can be between, for example, a transcription factor and a cis regulatory sequence or module as well as between signal transduction molecules or messengers and transcription factors, cis regulatory sequences, cis regulatory modules and any combinations thereof. The binding event can result, for example, in activation (e.g., positive effectors), deactivation (e.g. repressors), augmentation, or repression of the bound target network element or gene controlled by the bound target network element. Network interactions can be directional, reversible or essentially irreversible, for example, one or more series of connections, exemplified by circuits and sub-circuits, the various cis regulatory network elements serving as nodes in circuits and sub-circuits, and such with active cis regulatory connections maintaining a relationship of common function.

Accordingly, a “series” of cis regulatory network interactions is intended to mean a directional flow of two or more binding events between network elements. Such binding events can occur, for example, in contiguous or sequential spatial or temporal order within a cis regulatory network. Alternatively, element binding interactions can occur spatially or temporally non-contiguous or non-sequential within a network.

As used herein, the term “cis regulatory module” is intended to mean a collection of cis regulatory nucleic acid sequences elements that form a cis regulatory domain of a gene. Nucleotide sequences of cis elements that confer binding activity for a transcription factor or signaling factors constitute a cis regulatory nucleic acid sequence element. Combinations of such elements strung together constitute cis regulatory modules. Higher order combination (e.g., more elements) impart additional regulatory complexity and specificity onto their associated gene allowing diverse combinations of input signals to differentially control either the spatial or temporal expression or both of the associated gene. Cis regulatory sequence elements and modules are well known to those skilled in the art and can be found described in, for example, Kirchhamer and Davidson, Development 122:333-346 (1996); Yuh and Davidson, Development 122:1069-1082 (1996) and Davidson, E. H., Genomic Regulatory Systems: Development and Evolution Academic, San Diego (2001).

As used herein, the term “differentiation state” is intended to refer to the active or inactive set of genes within a given regulatory state. Such genes operate, for example, to control one or more function of a cell or group of cells in a particular regulatory state. A differentiation state therefore describes the relationship of genetic regulatory elements and the products they control in a cell at a given cellular state. Similarly, the term also can describe an active set of genes within regulatory states over a period of time including, for example, changes or relative differences between gene levels, activities or both. Therefore, the term can include, for example, temporal order as well as spatial order of gene set activity. Active genes can correspond to, and be determined by, for example, gene expression levels or rates; gene product levels, activity, or synthesis rates; or a combination of such measurements. Other measurements or attributes of gene activity or function well known to those skilled in the art can similarly be used to indicate an active gene set or relationship within a given regulatory state. Active genes of a regulatory state can include, for example, those genes or sets of genes (e.g., differentiation batteries, morphogenetic cassette) that control a particular cell function.

As used herein, the term “exogenous” used in relation to a transcription or signaling factor is intended to mean that the referenced cis regulatory network element or encoding nucleic acid originates or is introduced from outside of the endogenous cis regulatory network, genetic regulatory architecture, cell, tissue or organism. The exogenous network element or encoding nucleic acid thereof can be either heterologous or homologous, in relation to the cell tissue or organism of the network element, to the referenced cis regulatory network, genetic regulatory architecture, cell, tissue or organism. The term includes derivatives thereof, such as heterologous element to confer a new component activity into a cis regulatory network, genetic regulatory architecture, cell, tissue or organism, in combination with a homologous element. The introduction of a homologous element can be used to confer, for example, either a new component activity which is not currently present in the referenced environment or to confer an increased amount or activity of an already present endogenous element onto a referenced environment. In contradistinction to an exogenous network element or encoding nucleic acid an endogenous network element or encoding nucleic acid will already be present in the reference environment.

As used herein, the term “genetic regulatory architecture” is intended to mean the organizational structure of elements and the connections between them within a cis regulatory network. A genetic regulatory architecture represents, for example, an arrangement such as a network topology, wherein the binding activities, connections and resultant functions or gene products of a collection of interrelated transcription factors and their cognate cis regulatory nucleic acid sequences or modules are linked as performing a common function. The organizational structure can contain a single or multiple cis regulatory networks, organized as a plurality of nodes for which connections between the nodes establishes the network topology of circuits and sub-circuits. Therefore, a genetic regulatory architecture can represent any cis regulatory network including, for example, a cis regulatory network of a cell, tissue or organism, a cellular state, or a differentiation state. Similarly, the term “regulatory state” and “genetic regulatory state” refers to active set of genetic connections of cis regulatory nucleic acid sequences or modules and transcription and signaling factors in a cell. A regulatory state therefore describes the relationship of genetic regulatory elements in a cell at a given cellular state and thus delineates a genetic regulatory architecture of a cell at a point in time. The term also can describe a genetic regulatory architecture of a cell or group of cells over a period of time and can include, for example, changes or relative differences between elements or cell states. Therefore, the term as it is used herein can include temporal order as well as spatial order of active connections within a genetic regulatory architecture. Active connections can correspond to, for example, the expression level or rate of synthesis of a transcription factor, the binding activity of a transcription factor to a cis regulatory sequence or module, or a combination of such measurements. Other measurements or attributes of active genetic connections well known to those skilled in the art can similarly be used to indicate connectivity between elements of a regulatory state. Transcription factor and signaling elements of a regulatory state can include, for example, exogenous factors, such as those derived from an external signal or source, or endogenous factors, such as those that result from a genetic connection which activates or represses production of a transcription factor element.

As used herein, the term “network element”, and “cis regulatory network element” are used for a molecular constituent of a cis regulatory network. Such molecular constituents include, for example, polypeptides, such as transcription factors, signaling factors, nucleic acids, such as cis regulatory sequences and modules as well as other macromolecules or biochemical molecules that are constituents of a biochemical system such as a cis regulatory network. A plurality of such network elements can be represented in a network topology, by, for example, one or more nodes or edges conveying the identity of the component, binding connectivity, functional, spatial, temporal or other directionality and activity of the element. Other functions, characteristics and attributes of the network element can additionally be incorporated into the representation of the element depending of the desired need or use of the cis regulatory network.

As used herein, the term “regulatory territory” is intended to mean a spatial, temporal or functional category of a cis regulatory network. A regulatory territory can be, for example, either intracellular or intercellular.

### Overview of In Vivo Cell Reprogramming.

As described, cell fate is not fixed and one cell type can be converted into another as a product of specific regulatory processes encoded in the genome. Every function and every property of every cell is ultimately determined by the regulatory states generated by its genetic regulatory architecture, and gene regulatory network (GRN) theory allows for hierarchal organization of regulatory processes to establish network topology of cis regulatory network elements, such as transcription factors. Underlying the study of GRNs is the demonstrated principle that genetic programming for organismal development and cellular differentiation is hardwired and encoded in genomic DNA. The encoded genomic DNA provides a complex, yet organized network topology of cis regulatory network elements which can be described as circuits and sub-circuits. Identification of particular sub-circuits for targeted alteration allows for guided cell reprogramming in a starting cell type for generation of a transformed cell type by unleashing the encoded genomic program, and associated regulatory architecture governing the regulatory state in the transformed cell type. Importantly, in vivo or in situ cell reprogramming can be achieved when protein-based reprogramming factors are used, and this approach has wide appeal in treating difficult diseases.

Importantly, a key aspect of the claimed invention is that the encoded genomic program includes instructions for making virtually all cell types as a property of their genetic regulatory architecture, as the encoded genomic program is present in every cell. A thorough understanding of the genomic regulatory program controlling given cell differentiation processes allows one to reactivate the complete cellular differentiation program of a desired target cell type in a starting cell type. This is contrast to directed differentiation techniques in pluripotent and progenitor stem cells wherein morphogenetic factors and culture conditions only modify discrete elements within the starting cell type's regulatory architecture, without a view towards the reaching the complete genetic regulatory state of the transformed cell type. Use of the encoded genomic program offers a guided approach to cell transformation, without the laborious trial and error approach commonly associated with other directed differentiation methods. In particular, there are several advantages to protein-based in vivo cell reprogramming including safety leave no marks on cell genome, easier to deliver than genes or cells, more specific than small molecules, established fda regulatory path, along with experimental advantages related to flexible testing of reprogramming conditions (combination, time, dosage, and stoichiometry. In short, protein-based drugs described herein provide cell-based mechanism of action.

### Gene Regulatory Networks, Generally.

Gene regulatory network (GRN) models formalize the manner in which specification of cellular domains during development is controlled by spatial and temporal gene expression. It is now well-established that cell fate depends on expression of a specific set of regulatory genes, that is, genes encoding transcription factors and signaling factor molecules. In each domain of the developing organism and at each point in time, the genetic activities and therefore the fates of the cells are directly determined by the regulatory gene product and associated architecture present in the nuclei. The regulatory states constituted by these regulatory gene products are themselves the output of transcriptional control systems encoded in the genome. Such transcriptional control systems may be exemplified as a series of circuits, composed of sub-circuits, the network topology of which defines the identity and functional properties of the cell.

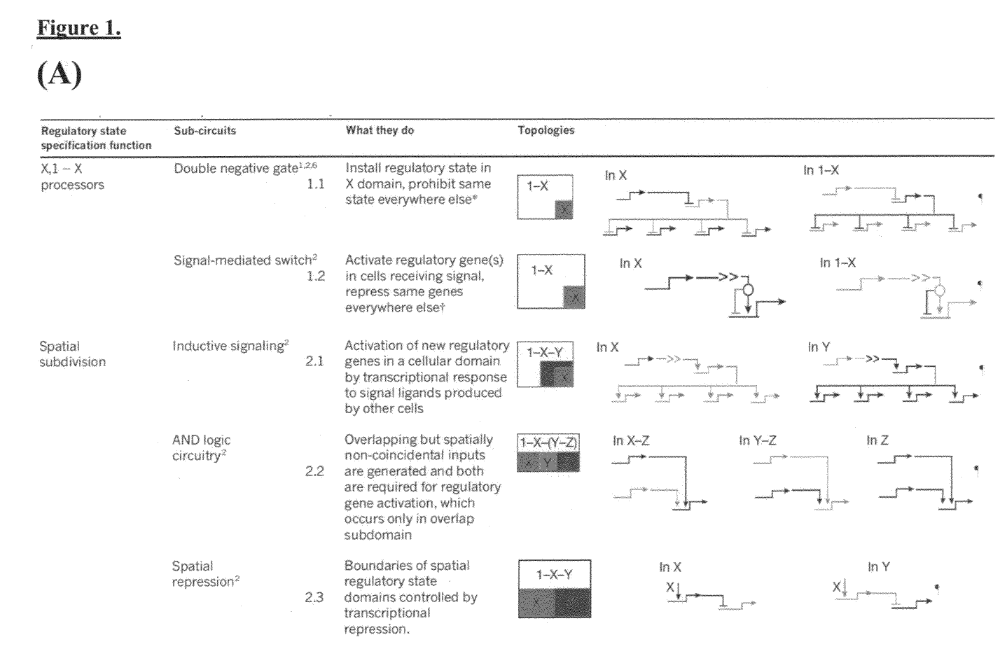

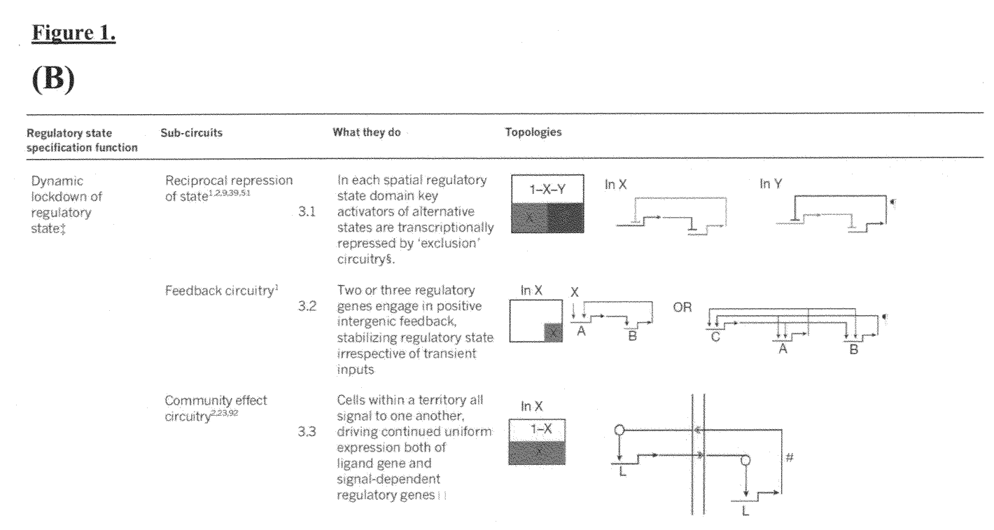

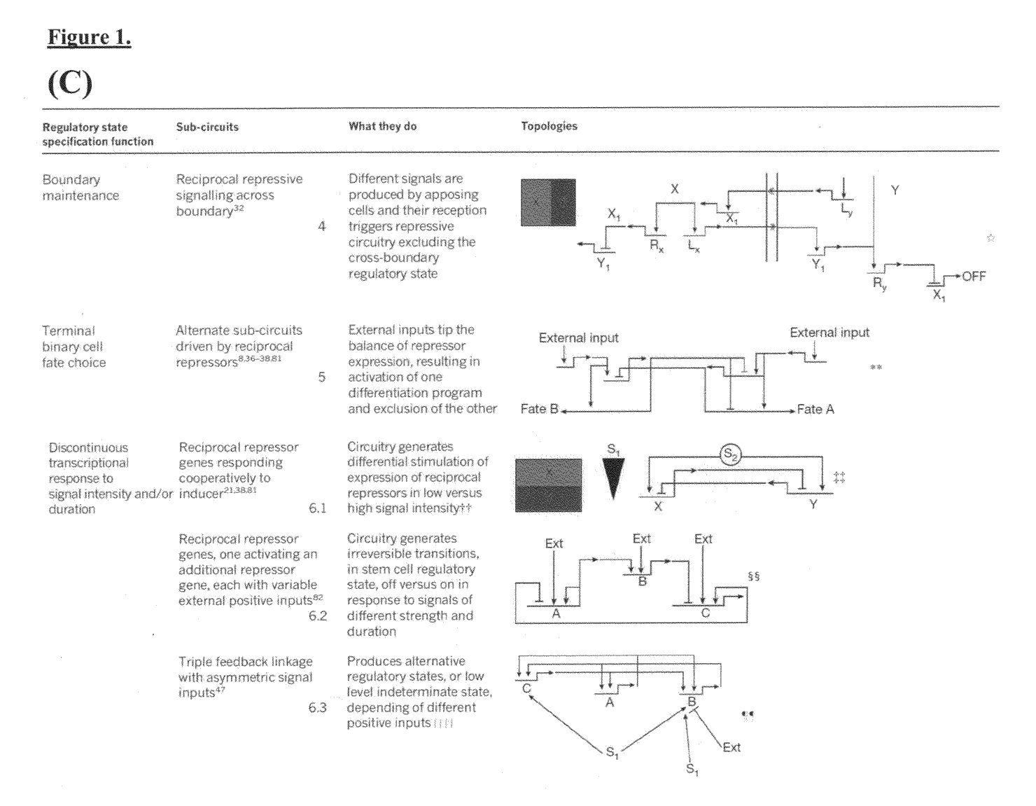

As an analytical approach, GRN thus recognizes the encoded genomic program for a regulatory architecture and captures the transcriptional control functions that specify various genetic regulatory states of the cell. One example includes the spatial regulatory states of the embryo. Such regulatory network architecture consist of regulatory genes and the transcriptional interactions that determine their specific patterns of expression. Models may be derived from experimental studies of developmental GRNs help to establish a link between genomic regulatory sequence as related to developmental process, and it is clear that this network toplogy offers a series of circuit and sub-circuit motifs arising from the regulatory architecture. A wide variety of these well-established network motifs is provided in FIG. 1. Under such regulatory architectures, every node in such network topology model represents a regulatory gene, which itself can be controlled by interactions encoded in genomic cis-regulatory binding sites to establish the genetic regulatory state of the cell. In this regard, GRN models represent syntheses of experimental gene expression and cis- and in many instances, trans-perturbation data, as aided by our understanding of observed developmental process.

Ultimately, a key aspect of GRN is a heterogeneous conglomeration of empirically established or predicted interactions. Importantly, if all regulatory genes and their interactions are known for a given process, network topologies can be constructed that will causally explain each gene expression event as the outcome of the preceding regulatory states, circuits and sub-circuits within the network topology of the regulatory architecture in a starting cell type can then be modified in order to generate a transformed cell type possessing a specific genetic regulatory state.

As large regulatory control systems organized as genetic networks, the lines of causality can be mapped from the genomic sequence to major processes of development and cellular differentiation. While a high degree of complexity exists for the regulatory architecture, particularly for body plan development and early developmental processes, there is clearly an established series of circuit and sub-circuit motifs arising from the regulatory architecture which can be modified for generation of transformed cell types.

The heart of organizing such cis regulatory networks includes: 1) genes encoding transcription factors or signaling factors and 2) the cis regulatory elements that in a positive or negative direction control the expression of those genes. Each of the cis regulatory elements receives multiple inputs from other genes in the network, the inputs being transcription factors which bind to a specific element that contains a specific cis nucleic acid sequence target sites. Functional linkages of which the network is composed are those between the outputs of regulatory genes and the sets of genomic target sites to which their products bind. These functional linkages which orchestrate in both a spatial and temporal fashion the differentiation fate and development plan of a cell or organism establish a network topology that can be analogized to electronic circuitry, its associated switches, capacitors and resistors. A variety of examples are well-understood and presented in FIG. 1. In this aspect, various transcription factors or signaling factors are nodes in a network topology organized by these functional linkages as organized by the inputs and outputs of regulatory genes.

### Deep Structure of Embryonic GRNs.

The most profound evidence supporting vitality of GRNs models is the de novo formation of embryonic territories, which typically include many different functional circuits and sub-circuits governing successive “layers” of process that are hierarchical in their overall structure. Here, the depth of embryonic body plan development reflects a long sequence of regulatory steps required to complete any component of embryonic development. In this regard, it is noted that GRNs may be approximated as deep or comparatively shallow structures, as characterized by the number of successive changes in regulatory state required to generate an episode of embryological or other development, between the initial state, terminal process, and final state which the GRN results in. That terminal outcome is driven by the activation of cohorts of effector genes (e.g., differentiation and cell biology genes, as opposed to only regulatory genes). In relatively shallow GRNs, some of which are considered below, the initial state may be a paused regulatory condition just upstream of expression of a differentiation gene battery. Thus, the concept that the position of target gene expression is determined solely by a quantitative value of a “morphogen”, as may be exemplified by a variety of ESC or iPSC studies resulting in “differentiated cells” of a particular cell type, may be overly simplistic. The overall pattern, and the overall signal strength response mechanism, are actually network properties rather than a property of individual cis-regulatory modules that independently and quantitatively read single gradient values.

Postembryonic Developmental GRNs: Differentiation from Pluripotent Stem Cells.

A remarkably recurrent similarity in GRN circuit design has recently emerged in studies of the transcriptional pathways that control binary fate choices executed in the diversification of specific cell types, such as haematopoietic cell types from multipotent precursors. At the cores of these circuits, which use some overlapping and some lineage-specific regulatory genes, are pairs of genes encoding transcription factors that mutually antagonize each other's expression within the same nucleus.

Often initially co-expressed at relatively low levels, the lineage fate choice depends on stepped up asymmetric expression of one or the other of the core repressor gene pair. Each of these genes also directly or indirectly promotes expression of positive regulators necessary for execution of one of the lineage fate choices. As the activity of one of the core repressors increases, it causes transcriptional extinction of expression of the alternative choice, and the irreversible installation of its own positive regulatory state. An important point is that the genes of the antagonistic repressor pairs, and/or the regulatory genes that are their immediate targets, also provide direct positive or negative inputs into terminal differentiation genes of the alternate lineages. In other words, this apparatus is deployed immediately upstream of the drivers of the effector genes that generate the features of given cell types. In comparison to the embryonic GRNs just considered, these are relatively shallow networks.

Ultimately, these types of genetic regulatory architectures rely on inputs into one or the other of the core repressors that can rely on extrinsic signaling ligands, for example cytokines and growth factors, including Notch and Tgfb, or endogenous immune receptor signals. The binary choice transcriptional apparatus responds to signal intensity, so that a low input gives one result and a high input another. Different pairs of repressor genes perform similar roles in different lineage fate choices, but what is remarkable is the similar circuitry adduced throughout, in for example, haematopoietic diversification. Transcriptional balance between pairs of cross-antagonistic repressors decides the outcome, for instance, in myeloid progenitors giving rise to macrophages or neutrophils; in precursors that may give rise to either B cells or macrophages, where there is cis-regulatory evidence of the transcriptional cross-repression; in the upper level decision point where erythroid versus myeloid fates bifurcate; in the erythroid versus platelet fate decision. Similarly, in T-cell diversification between helper vs killer fate, T-cell receptor signal strength indirectly controls repressor function, a compelling case because there is direct cis-regulatory evidence of the reciprocal transcriptional silencing interactions.

Although to some it is tempting to view all development through the same lens, there are fundamental differences between the terminal fate choice circuitry discussed here and the GRNs that execute early and mid-stage embryonic development of animal body parts. Differentiation gene batteries are activated only at the end of the series of GRN transactions that decide exactly where they are to be deployed. These cell fate decisions, such as hematopoesis, occur at the end of a complex prior developmental process, and in fact as discussed below, the circuitry controlling very early haematopoietic stem cell pluripotentiality operates in an entirely different manner from the binary choice circuitry just considered. In their function, haematopoietic binary choice sub-circuits are similar to the terminal sub-circuits that elsewhere in development immediately determine deployment of differentiation gene batteries. This perhaps explains why a characteristic of the stem cell differentiation choice systems, in other words the simultaneous low level expression in the multipotent precursors of differentiation genes indicative of multiple possible fates (e.g. lineage priming), is not seen in embryonic fate choices. That is, in embryonic body part development the spatial fate decision is made far up in the GRN hierarchy, and locked down, long before the differentiation gene battery is deployed. In contrast, in the production of functional immune cell types the last steps in the decision have to be deferred until the multipotential cells can be told which of its potentialities is more needed. Similar binary choice circuitry is also used in non-haematopoietic developmental contexts, but again at late stages in a given process where a terminal fate choice is to be made.

These kinds of sub-circuits, operate to choose, and/or to maintain the choice, of one of an alternative pair of differentiation gene driver sets. A priori, development of the body plan cannot be reduced to differentiated cell type specification, the last step in the process, nor to binary decisions between alternative fates. This is at root because development of the body plan requires a long sequence of multidimensional spatial decisions: during pattern formation spatial regulatory states must be installed progressively within multiple diverse boundaries, and also in certain anterior-posterior and dorsal-ventral positions with respect to the body plan. In each structure of the body regulatory states that include differentiation gene battery drivers are finally installed.

In view of this network topology placing differentiation gene batteries at the end of a long series of exchanges for body plan development, if the set of differentiation gene battery regulators is changed by experimental intervention, a different cell type can be made to appear. This is a key concept establishing the modularity of regulatory architecture within a starting cell type allowing for its exchange or intervention towards that of a transformed cell type.

Many recent studies have supported this concept by demonstrating that insertion of vectors expressing sets of transcription factors or even single transcription factors can result in the change of differentiated state from one haematopoietic cell type to another; from fibroblast to neuron, from exocrine to pancreatic b cell, etc. Again, it is emphasized that these cell fate changes all occur near the far downstream periphery of GRN hierarchy. Whereas growing a new body part requires a prior process of spatial pattern formation driven by a deep GRN, growing a new cell type simply requires activation of a new differentiation battery. More generally, although there are embryonic processes that look superficially like the binary choices just discussed, they are effected very differently.

As one example, in the sea urchin embryo, endomesodermal precursor cells give rise both to mesoderm and to endoderm, fates driven by entirely distinct regulatory states. But a careful experimental analysis shows that there is no pluripotential ‘endomesodermal’ GRN, and instead a Delta/Notch signal activates a set of regulatory genes which constitute a mesoderm GRN, while in the same cells a Wnt/Tcf signal activates a different set of regulatory genes which constitute the endoderm GRN. The genes of the mesoderm GRN and of the endoderm GRN are expressed independently of one another, without any interactions. The cells of each regulatory state are then separated physically by a cell division, so that the Notch signal is received exclusively by one ring of cells, which becomes mesoderm, while the other cells express the endoderm GRN exclusively. Nor are the exclusion functions that in given regulatory states act to repress genes key to alternative regulatory states ‘bipotential switches’. These sub-circuits are used to lock down regulatory choices already installed rather than to make choices. They may look superficially like the mutual repression sub-circuits that switch lineages bipotentially, but they are not. As related to various applications described herein, distinguishing between the circuits and sub-circuits related to body plan development and embryonic development, compared to later events of deploying differentiation gene batteries of morphogenetic cassettes can be evaluated when referring to the hierarchal organization provided by the network topology of a cell, its associated genetic regulatory architecture and desired genetic regulatory state.

### Differentiation Gene Battery Structure.

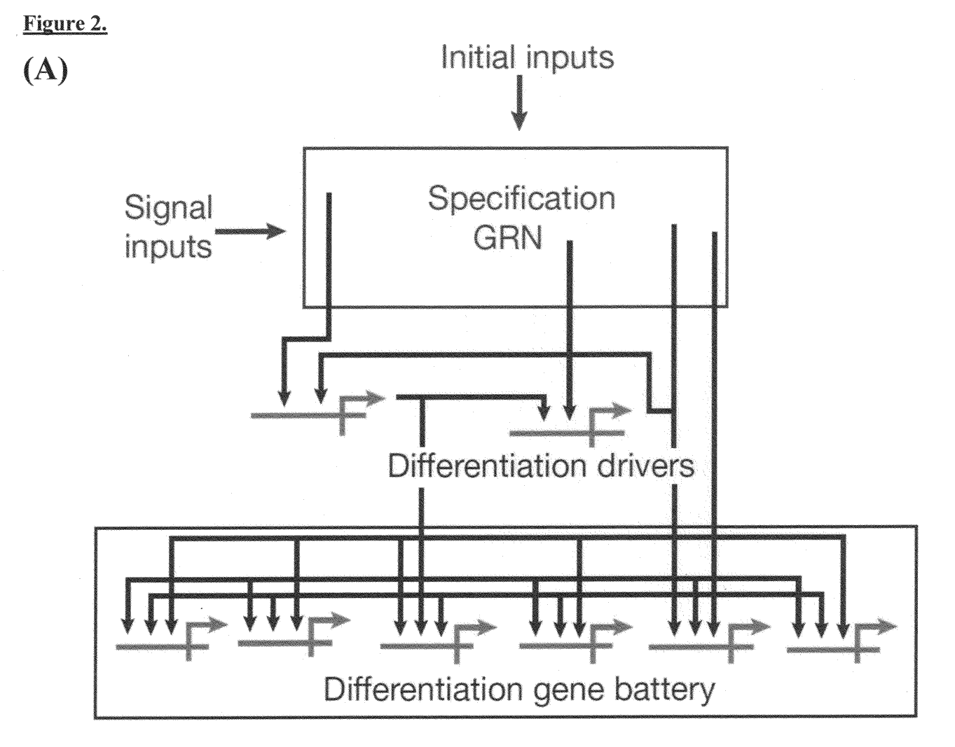

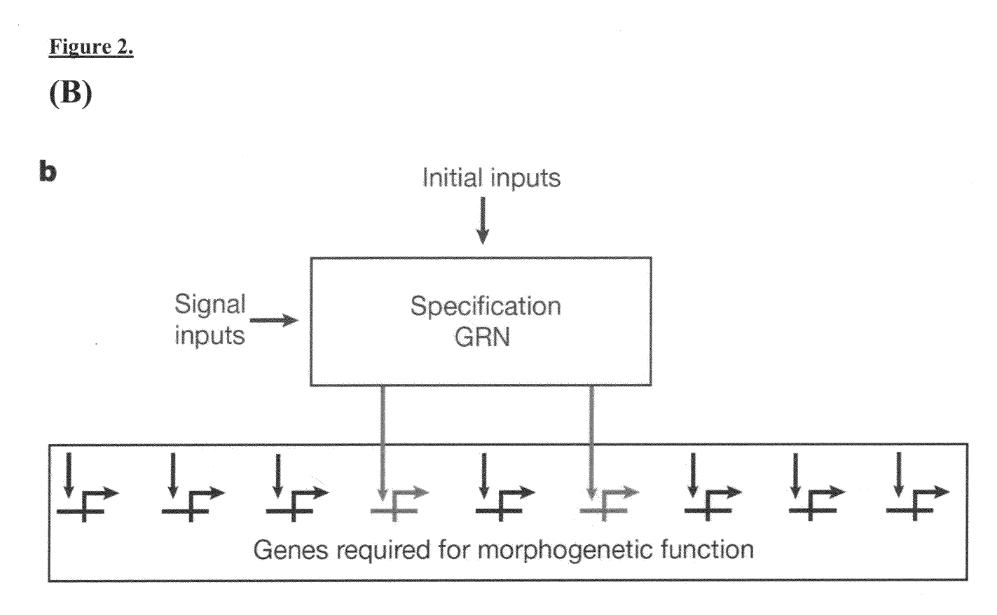

Differentiation gene batteries account for functional cell type specificity, and the comparatively shallow regulatory architecture readily identifies a series of network motifs sub-circuits that can be associated with them. Examples of the relevant network topology is presented in FIG. 2. This regulatory architecture readily presents a network topology of a finite number of regulatory relationships causing the protein coding differentiation genes of the battery to be expressed more or less coordinately.

In some instances, differentiation gene batteries may be relatively simply constructed types of sub-circuit, such as the coherent feed forward format, for which multiple examples can be found in sea urchin embryos, pancreatic b-cells, and macrophages. Importantly, improved understanding of the upstream GRNs clearly identifies an additional characteristic of differentiation gene battery regulatory circuitry: the occurrence of feedback between the drivers of the differentiation genes just upstream of the linkages to the effector genes, either auto- or cross-regulatory. While the ultimate breadth of differentiation gene batteries can consist of a very large number of effector genes, the relevant cis-regulatory modules of which (per battery) can actually respond to members of a small set of transcription factors present as part of the terminal regulatory state. In some instances, cis-regulatory module may in addition be serviced by some additional factors, accounting for the fact that all the genes of the battery are not exactly expressed in lockstep. For example, muscle protein genes are activated by two or three of the transcription factors orthologous to Srf, Mef2, and a myogenic bHLH factor in vertebrates, plus, individually, other factors; whereas in C. elegans the differentiation genes of each class of neuron are identified by their response to a single key transcription factor, sometimes together with other factors.

It is logically consistent that where there is direct repression of differentiation gene batteries by a proximal control circuit (‘anti-differentiation’) much the same architecture would be employed. In embryonic stem cells a hierarchical GRN that maintains the pluripotent state is headed by a recursive triple feedback system that links Nanog, Oct4 (also known as Pou5fl) and Sox2 genes. Apparently directly downstream of this are linkages to many genes encoding transcriptional activators and repressors, including a polycomb repressor that in turn targets regulatory genes associated with various differentiation states. But also among the immediate targets of the triple feedback loop is the Rest gene, which encodes a factor that directly represses neurogenic differentiation genes. This circuit is the mirror image of gene battery activation circuits.

### Structure/Function Relations for GRNs Controlling Diverse Kinds of Cell Biology.

The downstream effector gene cassettes required for development include those executing morphogenetic cell biology functions, as well as differentiation gene batteries. A distinction is that by definition, differentiation genes are expressed cell type-specifically, whereas genes required for functions such as motility, ingression, invagination, cell division, convergent extension, tube formation, branching, shape remodelling, epithelial-mesenchyme transition, etc., may be deployed in many diverse cell types and many diverse contexts in development. Examples of the relevant network topology is presented in FIG. 2.

Given the relatively close temporal and spatial proximity of differentiation gene batteries morphogenetic gene cassettes for specific cell biology, of interest is understanding their associated network topologies. One possible clue comes from various studies on GRN linkages that execute transcriptional control of cell replication in developing systems. The spatial patterns of cell replication of course affect morphology, because the size and shape of given portions of a structure depend on the number of rounds of cell division mediated by the regulatory state in each developing region. In several cases the exact outputs of a developmental GRN that specifically control cell cycle activity have been determined.

For example in developing pituitary, several linkages from the specification GRN directly control proliferation: the Pitx1 gene provides inputs into the cyclin D1 gene; the Six1 gene acts to repress expression of a cell cycle arrest kinase; and Six 1 plus other factors of the pituitary regulatory state activate c-myc (also known as Myc). In the developing zebrafish eye the GRN linkage to cell cycle control is regulation of cyclin D1 and c-myc (also known as myca/mycb) by the meis 1 regulatory gene. Thus, so to speak, these GRNs deploy the complex process of cell division by pressing a small number of regulatory ‘buttons’. Perhaps only a subfraction of the effector genes in a morphogenetic gene cassette are transcriptionally regulated by direct inputs from the upstream GRN. This concept emerged from a study of the migration of heart precursor cells in developing Ciona, one of the few system-level investigations we have into the transcriptional control of a morphogenetic function. A large number of cell biology genes participate in the processes of membrane protrusion and motility required for heart cell migration, but most of these genes are widely expressed. Migratory activity is specifically deployed by transcriptional activation of the rhoDF gene, which encodes a key required GTPase, and it is this gene which is directly controlled by the cis-regulatory outputs of the upstream GRN. The same principle is evident in a study of trichome formation in Drosophila. Here again, an extensive patterning GRN lies upstream, and determines the location of the morphological features and its cellular progenitors. The remodelling of epidermal cell shape to produce trichomes (or alternately, smooth cuticle) is controlled by expression of the regulatory gene shavenbaby (also known as ovo), and some of its direct effector gene targets are known. But these are again only a fraction of the total genes whose products are required to build the trichome.

With these examples as a guide, the wiring of differentiation gene batteries, in which every downstream gene is a specific target of the GRN, is distinct from the way morphogenetic gene cassettes may be wired. Many of the genes contributing to a morphogenetic cell biology process may be widely expressed and only a few key ‘button’ genes that functionally nucleate the whole process are transcriptionally controlled by GRN outputs, to deploy the process spatially. As a general result, this approach identifies existence of simple regulatory levers by which morphogenetic cassettes could be re-deployed, either in evolution or in re-engineering projects.

### GRNs: Mapping of Cis Regulatory Networks.

A network topology specifying the genetic regulatory architecture of a cell or cellular state can be depicted or conceptualized as a wiring diagram analogous to electronic circuitry, wherein various cis regulatory network elements are nodes whose organization are genomically encoded within a network, functional linkages between various nodes thereby organizing the circuitry, and sub-circuitry of the network topology. This cis regulatory network elements include for example, transcription and/or signaling factors, cis regulatory nucleic acid sequences, modules, related by binding activity and/or sharing a common function. Exemplary methods for determining the genetic regulatory architecture and deciphering the cis regulatory network for a cellular or regulatory state are described.

Briefly, the variety of methods involving a system analysis may include, for example, cis or trans perturbation of the cis regulatory elements, observation of developmental processes and the transcription factors involved in the cis regulatory network, identify functional regulatory linkages between cis regulatory elements, such as related transcription or signaling factors, and their associated cis regulatory nucleic acid sequences. In other instances, identifying the control elements and their target sites and then determining the functional significance of the linkage by any of a variety of methods well known to those skilled in the art. Cis regulatory analysis for functional determination of developmental and differentiation processes positions, a hierarchal organization of the regulatory inputs and outputs of the circuitry associated with the developmental and differentiation processes. It is worth emphasizing that a variety of network topologies have been established, example of which are known to one of ordinary skill including Davidson, The Regulatory Genome, Academic Press (2006), Faure, Emmanuel and Peter, Isabelle S. and Davidson, Eric H. (2013) A New Software Package for Predictive Gene Regulatory Network Modeling and Redesign. Journal of Computational Biology, 20 (6). pp. 419-423, Peter, Isabelle S. and Faure, Emmanuel and Davidson, Eric H. (2012) Predictive computation of genomic logic processing functions in embryonic development. Proceedings of the National Academy of Sciences of the United States of America, 109 (41). pp. 16434-16442, Peter, Isabelle S. and Davidson, Eric H. (2011) Evolution of Gene Regulatory Networks Controlling Body Plan Development. Cell, 144 (6). pp. 970-985. ISSN 0092-8674. U.S. patent application Ser. No. 10/746,277 Nam, Jongmin and Dong, Ping and Tarpine, Ryan and Istrail, Sorin and Davidson, Eric H. (2010) Functional cis-regulatory genomics for systems biology. Proceedings of the National Academy of Sciences of the United States of America, 107 (8). pp. 3930-3935, Davidson, Eric H. (2010) Emerging properties of animal gene regulatory networks. Nature, 468 (7326). pp. 911-920, Levine, Michael and Davidson, Eric H. (2005) Gene regulatory networks for development. Proceedings of the National Academy of Sciences of the United States of America, 102 (14). pp. 4936-4942, Istrail, Sorin and Davidson, Eric H. (2005) Logic functions of the genomic cis-regulatory code. Proceedings of the National Academy of Sciences of the United States of America, 102 (14). pp. 4954-4959. U.S. Pat. No. 8,178,347, each reference cited herein are incorporated by reference in their entirety as though fully set forth.

Functional linkages between plurality of nodes as regulatory inputs and outputs diagram the genetic circuitry. A diagram specifying cis regulatory connections irrespective of spatial or temporal activity describes the genetic architecture of functional linkages that are available to a cell or organism at any given point in differentiation or development. The genetic programming, through its cis regulatory network, turns on and off various circuits within this architecture throughout the development, differentiation and repair, remodeling or renewal processes to achieve precise biological outcomes.

For example, a wide variety of established circuits are depicted in FIG. 1, further explanation of which is provided here. Referring to item 1.1 of FIG. 1, double negative gates are “X, 1-X” processors that install regulatory state in X domain, prohibit same state everywhere else*. Signal mediated switches of item 1.2 of FIG. 1, are another form of “X, 1-X” processors that activate regulatory gene(s) in cells receiving signal, repress same genes everywhere else. Inductive signaling sub-circuits such as item 2.1 of FIG. 1, related to “spatial subdivision” by activating new regulatory genes in a cellular domain by transcriptional response to signal ligands produced by other cells. Logic circuitry is another form of “spatial subdivision” as shown in item 2.2 of FIG. 1, wherein overlapping but spatially non-coincidental inputs are generated and both are required for regulatory gene activation, which occurs only in overlap subdomain, and spatial repression sub-circuits show in 2.3 of FIG. 1 is another form of spatial subdivision, wherein boundaries of spatial regulatory state domains controlled by transcriptional repression. In addition, “dynamic lockdown of regulatory state” is a critical aspect of these processes, and sub-circuit repertoires are depicted. For example, reciprocal repression of state subi-circuit in item 3.1 of FIG. 1 depicts a sub-circuit whose role in each spatial regulatory state domain involves key activators of alternative states that are transcriptionally repressed by ‘exclusion’ circuitry. Likewise, a critical sub-circuit finding wide application in the claimed invention is, dynamic lockdown of regulatory state provided via feedback circuitry depicted in item 3.2 of FIG. 1, wherein two or three regulatory genes engage in positive intergenic feedback, stabilizing regulatory state irrespective of transient inputs, which of course can be expanded to ever increasing (e.g., four, five, six, seven or more) regulatory genes. Another example of dynamic lockdown of regulatory state is depicted in item 3.3 of FIG. 1, via community effect circuitry, this type of sub-circuit provides opportunity for cells within a territory all signal to one another, driving continued uniform expression both of ligand gene and signal-dependent regulatory genes. Another regulatory state specification function includes “boundary maintenance”, for which reciprocal signaling across one or more boundaries is a common motif, as shown in item 4 of FIG. 1. Here, different signals are produced by apposing cells and their reception triggers repressive circuitry excluding the cross-boundary regulatory state. In addition, “terminal binary cell fate choice” are a commonly and well-understood function in cells, by which sub-circuits such as alternate sub-circuits driven by reciprocal repressors depicted in item 5 of FIG. 1, allow external inputs to tip the balance of repressor expression, resulting in activation of one differentiation program and exclusion of the other. In addition, “discontinuous transcriptional response to signal intensity and/or duration” is a common cellular function, and the reciprocal repressor genes responding cooperatively to inducer sub-circuit depicted in item 6.1 of FIG. 1 demonstrates how this architecture generates differential stimulation of expression of reciprocal repressors in low versus high signal intensity. In addition another, discontinuous transcriptional response sub-circuit is depicted in item 6.2, that of the reciprocal repressor gene organization, with one activating an additional repressor gene, each with variable external positive inputs. Here, the circuitry organization generates irreversible transitions, in stem cell regulatory state, off versus on in response to signals of different strength and duration. Another example of discontinuous transcriptional response includes the triple feedback linkage with asymmetric signal inputs design shown in item 6.3 of FIG. 1, this design produces alternative regulatory states, or low level indeterminate state, depending of different positive inputs.

In contrast, a diagram or other compilation specifying those cis regulatory connections occurring at a particular time or place will describe the precise genetic regulatory state of functional linkages that are active or inactive during that point of the development, differentiation, repair, remodeling or renewal processes. In short, these are the genetic circuits that are temporally and spatially active within the organism or cell at the time of the monitored event. The composite of spatial and temporal connections active during a particular developmental, differentiation, repair, remodeling or renewal processes is one characteristic of the genetic regulatory architecture that specifies the regulatory state of the cell. In turn, a regulatory state is a regulatory fingerprint that characterizes or can be correlated with its corresponding phenotypic cellular state.

The interconnections specified in a cis regulatory network of the invention will consist of the binding interactions between the various network elements that are related by a common function. As described previously, these cis regulatory network elements will consist of transcription factors, signaling factors, and other described cis regulatory elements and cis regulatory modules. The binding interactions can represent any activity of the included network elements and include, for example, one or more transcription factors binding to one or more cis elements or modules to effect activation or repression of the bound cis sequence. Similarly, binding activities can be interconnected by sequential or parallel interconnections induced by one or more initial binding activities to represent a consequential series of binding interactions that have been induced or repressed by a referenced binding activity.

A cis regulatory network compilation also can include interrelationships within a common function other than those binding activities between transcription factors and cis elements or modules. For example, a cis regulatory network of the invention can further specify activities of inducers, inhibitors or other types of regulators that initiate from external origins relative to the cis regulatory network. Similarly, activities of inducers, inhibitors or other types of regulators exported from a cis regulatory network following production also can be specified in a cis regulatory network of the invention. Such inducers, activators or regulators can include, for example, hormones, growth factors, second messengers, signaling ligands, ligands, and cofactors. Further, gene products of other than transcription factors also can be included in a cis regulatory network of the invention as well as all types of macromolecules, and molecules when desired to impart information on the function or activity of a cis regulatory network.

### Transformation of Cell Fate.

As a widely sought after objective for regenerative medicine applications, multiple disease and trauma states could be alleviated if cell fate could be altered to a desired different cell fate in order to compensate for loss or dysfunction of endogenous cells. Via the described methods, GRN offers a rational approach to transformation of cell fate that in some instances, requires no more than a single application of exogenous gene regulatory proteins (“transformation motivator”) such that there are no insertions of exogenous genes to the genome, and such that the transformation motivator is present in the target cells only transiently. In this particular application, risk are eliminated for potentially dangerous and expensive aspects for introduction into the body of cells transformed in vitro, or of somatic genomic insertions. The focus of the approach is to elicit the genomic programming of cell type by activating the same endogenous regulatory functions as are normally activated in the course of development of the tissues which include the desired differentiated cell type. The transformation motivator acts as a trigger which animates the normal endogenous genomic instructions for development.

As related to selection and transformational motivator, if the endogenously encoded genetic regulatory architecture and its genetic regulatory state specific to a transformed cell type can be activated in a starting cell type, the genetic regulatory state will be transformed to that of transformed cell type. It is noted that beyond the desire for a particular feature of the transformed cell type if one can deploy the genetic regulatory state of the starting cell type is entirely and completely altered to that of transformed cell type, the functional properties of the cell, including both known and unknown features, will be entirely those of the transformed cell type since the genetic regulatory state ultimately controls all cell function. The genetic regulatory architecture as circuitry contains the information required to determine the content of the transformation motivator, such as that of the transformed cell type, so that if introduced into recipient starting cell type it will effect the permanent activation of the terminal differentiated genetic regulatory state of the transformed cell type.

The circuit elements of the GRN that indicate the composition of the transformation motivator are specifically those which encode self-sustaining dynamic transcriptional functions which in turn provide inputs into the remaining genes of the GRN that gives rise to the desired differentiated cell type. These are feedback circuits which cause the near terminal and terminal regulatory states to function independently of prior developmental regulatory inputs. This provides a rational index for transformation strategy. In some applications, positive feedback loops are particular useful for permanent and durable alteration following single introduction of an exogenous protein or proteins, and/or nucleic acids encoding such proteins, because if the transcription factors causing the activation of such feedback circuits are introduced these proteins themselves will no longer be required s they will have activated the genes of the feedback circuit, which will cause each other to generate their own transcripts. Thus, a transient application of a transformation motivator could generate a permanent new regulatory state.

Described herein is a method of transforming a cell, including providing a quantity of at least one cis regulatory network element and introducing into a starting cell type, the at least one cis regulatory network element, wherein the at least one cis regulatory network element is capable of altering a regulatory sub-circuit in the starting cell type, thereby altering one of more properties of the starting cell type, and generating a transformed cell type. In other embodiments, the cis regulatory network element includes a transcription factor and derivatives thereof. In other embodiments, the cis regulatory network element includes a recombinant protein. In other embodiments, the cis regulatory network element is encoded by a nucleic acid. In other embodiments, the regulatory sub-circuit is a positive feedback loop. In other embodiments, the regulatory sub-circuit includes at least two cis regulatory network elements. In other embodiments, the regulatory sub-circuit includes at least three cis regulatory network elements. In other embodiments, the one or more properties includes transcription factor expression and/or transcription factor binding to a cis regulatory network element. In other embodiments, the one or more properties includes protein expression and/or surface marker expression. In other embodiments, the starting cell type is a hepatocyte. In other embodiments, the starting cell type is a non-insulin secreting islet cell. In other embodiments, the transformed cell type is an insulin secreting islet cell. In other embodiments, the insulin secreting islet cell expresses Pdx, MafA and Ngn3. In other embodiments, the starting cell type is a peripheral T cell. In other embodiments, the transformed cell type is a T_{reg} cell. In other embodiments, the T_{reg} cell expresses Foxp3. In other embodiments, the starting cell type is a mesenchymal stem cell. In other embodiments, the transformed cell type is a chondrocyte. In other embodiments, the chondrocyte expresses Sox9. Further described herein is a quantity of transformed cells made by the described method.

Modulating a regulatory circuit or sub-circuit state in the starting cell type can include introducing one, two, three, four, five, six, seven, eight, nine, ten or more network elements into a cell, thereby altering one or more properties of the starting cell type, induce a predetermined change in the cis regulatory circuitry. In some designs a desired regulatory state can be achieved through introduction of only one network element such as a transcription factor or cis element or module. In other designs, achieving a desired regulatory state will require from several to many network element changes. The number of alterations in the cis regulatory network necessary to impart a particular function is determined, in part, by hierarchal organization when compared to the end result. For example, as described, those networks related to embryonic development, such as body plan organization, or early cellular differentiation are comparatively deep networks requiring a series of temporally, spatially, or otherwise described, distant or numerically greater events, whereas differentiation batteries and morphogenetic cassettes are relatively shallow, such as the positive feedback loops described in FIG. 1. Thus, the complexity of the regulatory network circuit or sub-circuit altered dictates the number of cis regulatory network elements that are introduced as based on the regulatory architecture of the starting cell type and its similarity or dissimilarity to the regulatory architecture of the transformed cell, the latter possessing the desired functions of the system. Importantly, the positive feedback loops described herein for a desired cell type is that the transformational motivator can result into a durable and/or permanent “lock on” state once the cells' fate is decided, which is maintained through several positive feedback loops or self-sustainable mechanism. Thus, unlike other recombinant expression systems, here, introduction of a select number of external key transcription factor proteins can initiating a new genetic regulatory stated in the transformed cell type, the external reprogramming factors may no longer be required once the a new GRN is initiated.

Once introduced, the cis regulatory network elements will perform their transcription regulatory functions by binding to or being bound by their cognate cis elements or transcription factors to initiate a series of cis regulatory network interactions. The series of interactions can be activation, repression or both activation and repression one, two, three, four, five, six, seven, eight, nine, ten or more network elements. The result of such interactions will be to produce a cell having the specified regulatory state of the underlying modified cis regulatory network. The cis regulatory element or module can be chosen to control expression of a transcription activator or transcription repressor. In some instances targeted knockdown, permanent or transient, of a particular node may be sought in view of its organization in the circuit or sub-circuit. Similarly, the activators or repressors can function to lock down and commit a regulatory state by causing the expression of positive effectors for a differentiation battery or morphogenetic cassette causing cohorts of genes in the battery or cassette to be express. In another instance, the alteration prohibits commitment to differentiation or development the cell type, such altering lineages and/or differentiated states, or imparting the changes in one or more properties of the starting cell via new expression of proteins, cell surface markers, functional properties in the transformed cell type. The introduction of a homologous element can be used to confer, for example, either a new component activity which is not currently present in the referenced environment or to confer an increased amount or activity of an already present endogenous element onto a referenced environment. In contradistinction to an exogenous network element or encoding nucleic acid an endogenous network element or encoding nucleic acid will already be present in the reference environment.

As described, the genetic regulatory architecture as a cis regulatory network includes a plurality of cis regulatory network elements, such as a transcription factor or signaling factor, operating as an activator or repressor, these various cis regulatory network elements thereby providing a plurality of nodes which can be organized in a network topology of circuits and sub-circuits. Alteration of the nodes in relation to the network topology of circuits and sub-circuits allows for alteration of one or more properties of the staring cell type, which results in the genetic regulatory state of the transformed cell type. For example, transcription factors can be chosen, to bind to a cognate cis element or module and modulate the expression of a linked regulatory gene. Similarly, the transcription factors themselves can be introduced and function as positive effectors in a positive feedback loop, wherein a first node, promotes the expression and regulation of as second node, the second node promoting the expression and regulation of a third node, the third node promoting the expression and regulation of the first and second nodes, and various combinations of these features. These positives feedback loops can durably and permanently commit the regulatory state of the starting cell type, thereby altering one of more cellular properties, and generating the transformed cell type. In addition to unleashing these differentiation batteries or morphogenetic cassettes to lock down a genetic regulatory tate in positive feedback loops, alternative designs may fully repress an alternative genetic regulatory state.

Various combinations of cis regulatory network elements can be applied in a network topology of circuits and sub-circuits to achieve a specified result. Depending on the underlying network topology, the transformational motivator or control drivers chosen for introducing into the starting cell type is designed in view of the plurality of nodes, organized in a network topology of circuits and sub-circuits, deployed upstream of circuit or sub-circuits for activate or inhibit downstream elements. Given the teachings and guidance provided herein, those skilled in the art will known or can determine the targeted node or nodes capable of altering a regulatory circuit or sub-circuit in a genetic regulatory architecture in order to confer the desired regulatory stated in the transformed cell. For example, the genetic regulatory architecture for the starting cell type that provides the causal linkages generating the regulatory state of the transformed cell type. In various embodiments, the regulatory state includes the sum total of transcription factors present in the nuclei of the target cell type. The genetic regulatory architecture, regulatory states of starting cell and transformed cell type is utilized as an instruction to choose a combination of transcription factors that can will, according to the circuit and sub-circuits in the network topology, regulate all other transcription factor genes required in this cell type and thus to efficiently and permanently transform other host cells to the target cell type. Importantly, inducing transcription of all transcription factor and signaling genes normally expressed by the transformed cell type (i.e. its normal genetic regulatory state). The expression of all genes in the starting cell type depends on the expression of its genetic regulatory state and thus will likewise be induced in transformed cells. In applying the method, one can begin by identifying of potent regulatory gene circuits consulting the hierarchal organization of the network topology, circuits and sub-circuits. Two helpful criteria to identify essential subicircuits in a GRN model firstly include genetic sub-circuits whose activation will maintain their expression. The major class of such sub-circuits include positive feedback loops, where the transcription factors encoded by the genes in the circuit activate each other's transcription. Additional circuit features contributing to the stability of these feedback loops is the existence of a common driver for the genes into the positive circuit. Secondarily, sub-circuit interconnection and operation at a higher level relatives to differentiation batteries or morphogenetic cassettes thereby causes expression of other transcription factors as regulated downstream of these sub-circuits. As the described positive feedback sub-circuits constitute cell-type switches that run permanently in that cell type, the described approach takes advantages of a genomically encoded program.

The orchestrated series of alterations focusing on nodes in the network topology implementing a particular genetic regulatory state will constitute different regulatory states of the cis regulatory network. Because a regulatory state also delineates a genetic regulatory architecture of a cell at a given point in time, the described network toplogy also will characterize the cellular state of the starting cell type and the resulting transformed cell type.

Alterations of a cis regulatory network to modify a genetic regulatory state of a cell can be performed in essentially any desired type of cell, tissue or organism. In essence, any cell or group of cells can be reprogrammed to generate a different cell of a desired regulatory state. By choosing an appropriate node or nodes capable altering genetic regulatory state, various differentiation batteries or morphogenetic cassettes be induced to confer alterations one or more properties in starting cell type, resulting in a transformed cell type. For example, progenitor cell also can be induced to change genetic regulatory states without altering its physiological differentiation or developmental characteristics. For example, an undifferentiated or less differentiated cell can be reprogrammed by introduction of cis regulatory network elements to differentiate. Conversely, a differentiated or more differentiated cell can be modified by introduction of cis regulatory elements to dedifferentiate.

Various types of cells that can be reprogrammed into a specified regulatory state include, for example, a pluripotent stem cell such as embryonic stem cells (ESCs) and induced pluripotent stem cells (iPSCs), a pluripotent lineage specific progenitor cell, a progenitor cell or a terminally differentiated cell. Progenitor cells used in the methods of the invention can be derived from any tissue harboring such cells. Further, given the available methods well known to those skilled in the art, a progenitor cell can be reprogrammed by either ex vivo, in vivo, or in situ. This includes techniques described in U.S. application Ser. No. 13/141,326, U.S. App. No. U.S. Ser. No. 13/288,040, PCT App. No. PCT/US2009/069518, PCT App. No. PCT/US2011/041709, PCT App. No. PCT/US2011/02 3259, PCT App. No. PCT/US2011/023259, and PCT App. No. PCT/US2012/047495, each reference cited herein are incorporated by reference in their entirety as though fully set forth. Cis regulatory network elements can be introduced, for example, into a single cell, a population of cells, cells within tissues, organs or organisms or whole populations of cells constituting a tissue, organ or organism. Those skilled in the art will known what format of element introduction is appropriate for a given application. For example, where a reprogrammed progenitor cell can be implanted or transplanted method for ex vivo modification can be used effectively. In contrast, in vivo or in situ modification can be effectively used where vectors and targeting moieties are available for the progenitor cell.

Further described herein is a method for identifying a regulatory network for transforming a cell including: organizing a plurality of cis regulatory network elements into a network topology of nodes including circuits, wherein the circuits include at least one sub-circuit, identifying at least one sub-circuit including at least one positive effector node, wherein the at least one positive effector node is capable of generating a transformed cell type when introduced into a staring cell. In other embodiments, the sub-circuit is a positive feedback loop. In other embodiments, the sub-circuit includes at least two cis regulatory network elements. In other embodiments, the sub-circuit includes at least three cis regulatory network elements.

For example, the genetic architecture of a cell is made up of a plurality of cis regulatory elements, which can be described as nodes, the plurality of nodes being further organized according to the functional linkages described, thereby establishing a network topology for the nodes, including circuits and sub-circuits. As described, the genetic regulatory state of the cell presents a landscape of regulatory genes whose architecture encodes circuits and sub-circuits for introduction into a starting cell type, wherein expression of the introduce cis regulatory network elements generates cells of the desired regulatory state, resulting in the transformed cell type. Modulation can be accomplished by, for example, using nucleic acid constructs encoding the transcription factor or signaling factors, or introducing of factors as exogenous protein for activating differentiation batteries and/or morphogenetic cassettes. The nucleic acid constructs can be controlled by, for example, inducible expression systems, constitutively active expression systems or expression systems that respond to regulatory inputs already present in the initially uncommitted cells or to regulatory inputs that become activated during development or differentiation. Alternatively, the modulation can be achieved by, for example, using upstream regulators of the identified regulatory genes such as hormones, growth factors and other cell signaling molecules or functional equivalents thereof. Modulatory nucleic acid constructs or exogenous proteins can be introduced into the uncommitted cells for expression of the encoded regulatory factors with concomitant steering of the cell down the intended developmental or differentiation pathway via activation of differentiation batteries or morphogenetic cassettes. Modulation of regulatory genes and cis elements also can be accomplished using, for example, various effector molecules known in the art such as exogenous proteins, RNAi, small molecule compounds and antisense nuclear acids. Turning on or off existing circuits, sub-circuits, bypassing circuits, sub-circuits or adding new circuits, sub-circuits allows control of differentiation, developmental, repair, remodeling or renewal processes for directing the process down a predetermined outcome or path.

Also described herein is a composition including a quantity of cells expressing at least one exogenously added protein, wherein the at least one exogenously added protein is a positive effector in a regulatory sub-circuit. In other embodiments, the cells express at least two exogenously added proteins, and the at least two exogenously added proteins are each positive effectors in a regulatory sub-circuit. In other embodiments, the regulatory sub-circuit is a positive feedback loop.

Also provided is a cell having a specified regulatory state, comprising a cis regulatory network having a modified genetic regulatory architecture, said modification comprising two or more exogenous transcription factors activating a predetermined series of cis regulatory network interactions, said series of cis regulatory network interactions resulting in a specified non-naturally occurring regulatory state of said cell.

Cells can be reprogrammed by design based on the genetic regulatory to generate a newly specified genetic regulatory architecture or to generate a newly specified regulatory state within an existing genetic regulatory architecture. The desired end point as a regulatory state will determine whether a new functional linkages between transcription factors and their cognate cis regulatory elements are required or whether activation, repression or both activation and repression will suffice to generate a desired regulatory state from the staring cell type. For example, if the interconnections specified in a genetic regulatory architecture exist, but are naturally regulated to prevent occurrence of a desired regulatory state, alterations via targeting of a node or nodes in relation to its circuit or sub-circuit can be performed to spatially or temporally change the sequence of binding connections to achieve the necessary functional linkages, such as binding events, for the required outcome. In this instance, an existing cis regulatory architecture is reprogrammed to achieve a different and specified genetic regulatory state in the transformed cell type.

Cells having a specified regulatory state produced by modification of a genetic regulatory architecture can be isolated, propagated, stored and manipulated by any of various methods well known to those skilled in the art. For example, following alteration of the circuits or sub-circuits in a starting cell type by targeting of a node or nodes in a network topology organized by the genetic regulatory architecture, cells can be isolated by culture, selection, fluorescent activated cell sorting (FACS) or other methods well known to those skilled in the art. The cells can be further propagated under appropriate culture conditions for the particular cell type generated or stored, such as by cryopreservation, for future use. Further, cells produced having a specified regulatory state can additionally be manipulated by genetic or biochemical methods well known to those skilled in the art. For example, the produced cells can be additionally modified by the introduction of nucleic acids encoding desired gene products for expression and polypeptide production either in vitro or in vivo. Essentially, all methods available to the skilled person in the fields of cell, molecular or developmental biology as well as biochemistry and physical chemistry are similarly applicable to cells produced by the methods of the invention. Similarly, methods of therapy, including cell therapy and transplantation, and diagnosis also are applicable to the cells produced by the methods of the invention. Accordingly, the cells produced by the methods of the invention are substitutable in methods well known to those skilled in the art.

### Example 1

### Applications of Genetic Regulatory Networks, Generally

As described, network topologies for various organisms in specific developmental or functional contexts have been established and one of ordinary skill can consult such references cited herein. For comparing the genetic regulatory architecture and genetic regulatory states of two different types of cells, many different transcription factors will differ significantly in expression. Among these various network topologies, such cis regulatory network elements, represented as a plurality of nodes operate and circuits and sub-circuits such as that depicted in FIG. 1. Importantly, the existence of this repertoire of circuits and sub-circuits demonstrates a high degree conserved of conversation, providing a hierarchal organization for which targeting of particular modules within the network topology allows for opportunity to alter properties of a starting cell type into a transformed cell type possessing desired properties. Some nodes within circuits or sub-circuits in the transformed cell type may be inactive in the starting cell type, but which the protein products of such nodes are instrumental in the transformation process, as leading to a cascade of subsequent protein expressions or cell fates via deployment of differentiation batteries or morphogenetic cassettes.

Here, specific examples are provided for adapting genetic regulatory network programming approaches to transform cells in a permanent and durable “lock on” state following selection of a transformational motivator or controlling driver activating positive feedback loops as a self-sustainable mechanism. Upon introducing several external key transcription factor proteins and initiating the desired genetic regulatory state, the external reprogramming factors may no longer be required as self-perpetuating, providing durable and permanent expression superior to other recombination expression approaches. Even when the reprogramming proteins are withdrawn from the environment, the cells will maintain the new fate. For each sub-circuit it is possible that introduction of a transformational motivator, or controlling driver transcription factor will suffice to start the endogenous target feedback circuit. Introduction of the driver factor plus one of the factors encoded by the endogenous genes of the feedback loop as suffice to ensure continued transcription of all three endogenous genes of each sub-circuit. Examples provided herein include transformation of liver cells to insulin secreting cells, transformation of non-islet cells of the pancreas to insulin-secreting cells, transformation of peripheral T cells to Treg cells, and transformation of mesenchymal stem cells to chondrocytes.

### Example 2

### Transformation Using β-Cell Regulatory Architecture

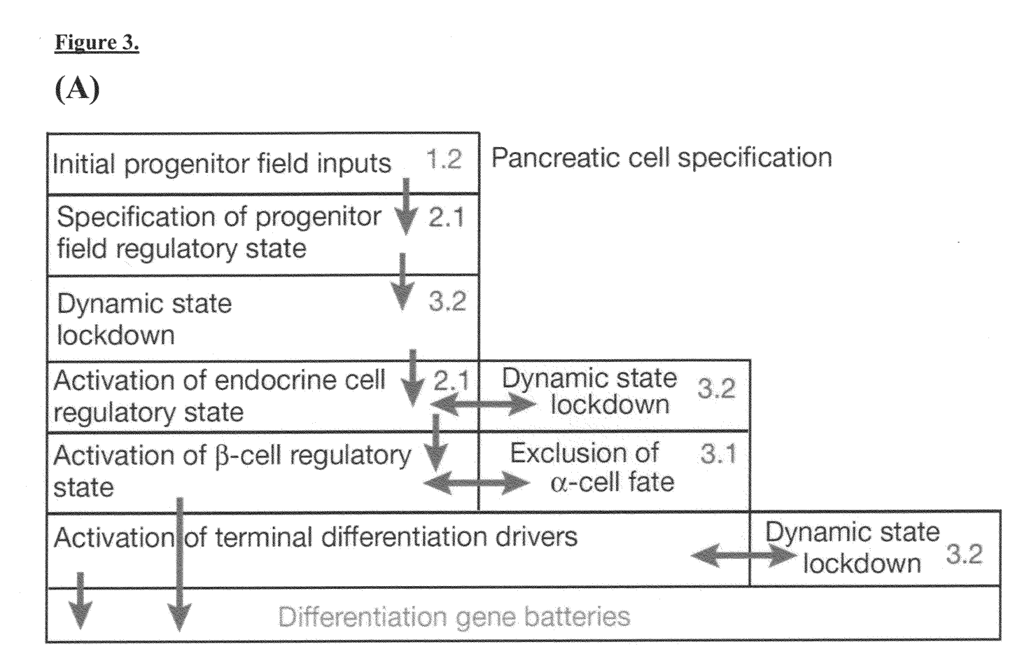

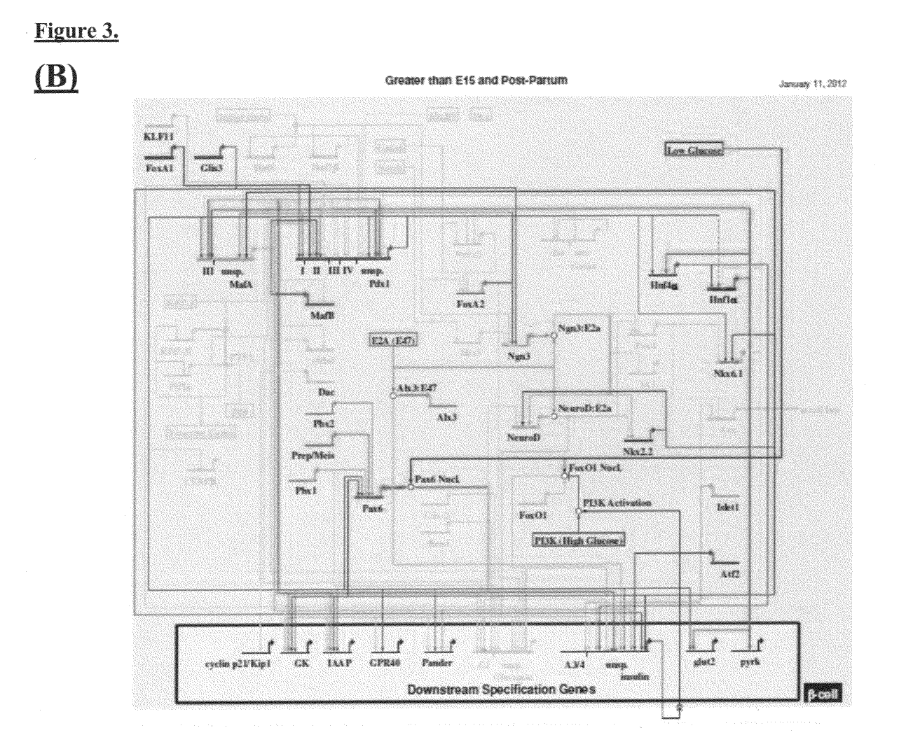

One example, the described pancreatic β-cell genetic regulatory architecture includes >20 regulatory genes expressed during last stages of development of insulin secreting cells. A general hierarchal organization and network topology is presented in FIG. 3(A). As with all cells, sub-circuits provide specific cellular functions, such as causing various endocrine genes to be active, repress genes of other cell types, account for responses to glucose, and other physiological responses; ensure stability of regulatory state. A complete network topology of β-cell is presented in FIG. 3(B).

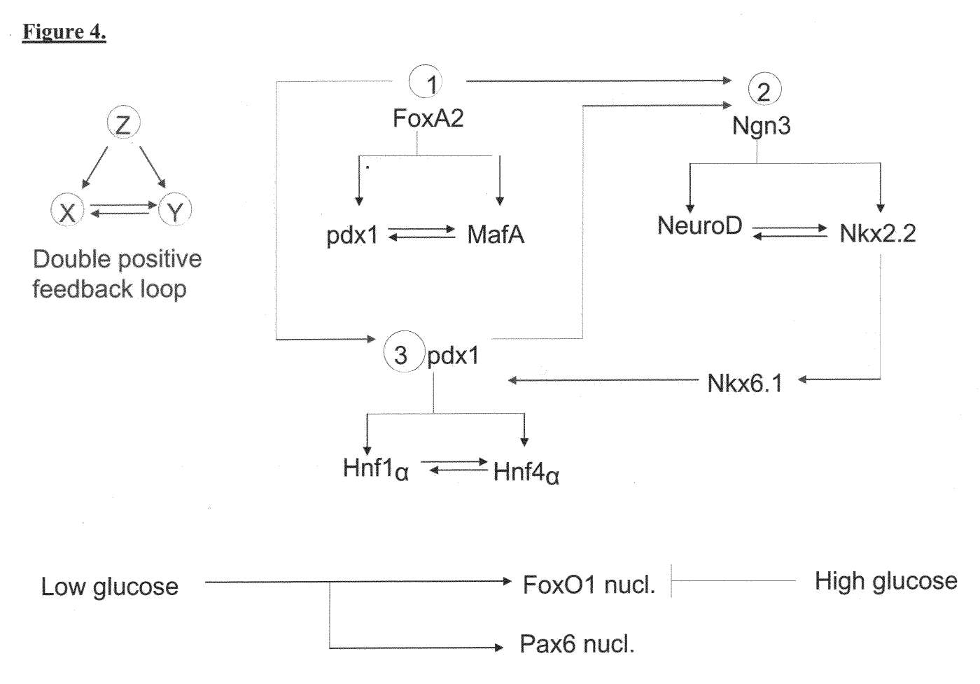

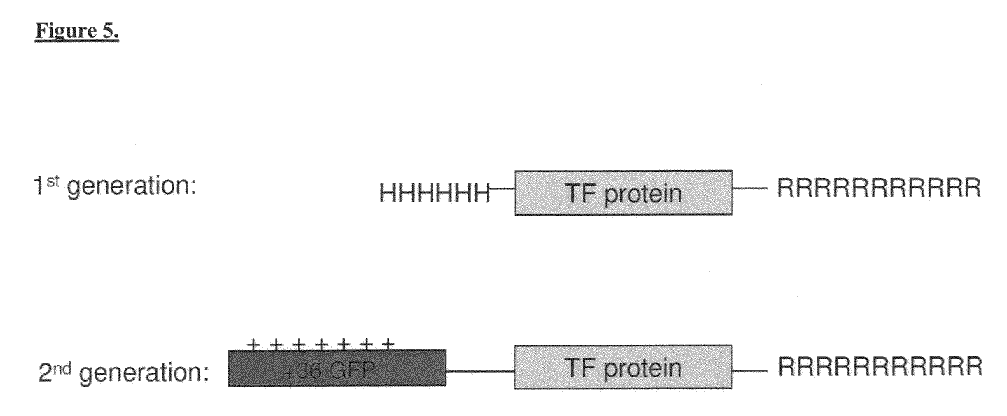

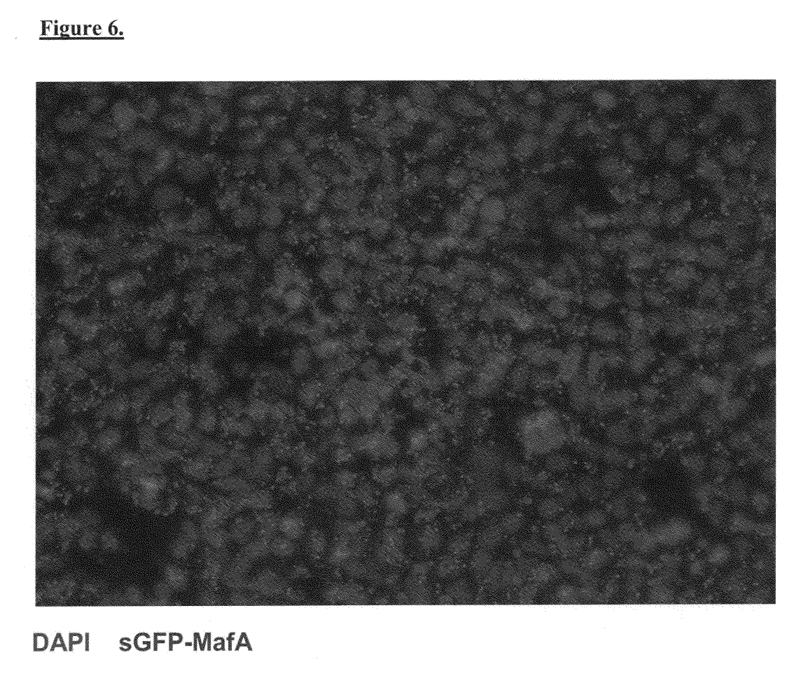

A vital aspect of using genetic regulatory structure to effect permanent transformation strategy include those developmental self-perpetuating feedback circuits that when activated permanently and durably lock down cell fate by driving the activity of rest of the genetic regulatory architecture conferring a particular genetic regulatory state. For example, a modular sub-circuit is presented in FIG. 4. These sub-circuits can be recognized by their structure they are encoded in the genomes of every cell. Once activated in a starting cell type, the self-perpetuating positive feedback loops should inherently elicit the genetic regulatory state of which they are the drivers, thereby conferring a transient regulatory intervention to produce a permanent change in cell fate and function. It is worth emphasizing that despite the complexity within the complete network topology of FIG. 3(B), identification and manipulation of a specifically identified sub-circuits allows widespread transformation of a starting cell type into the transformed cell type, thereby demonstrating the versatility and profound effects of a targeted, combinatorial approach.

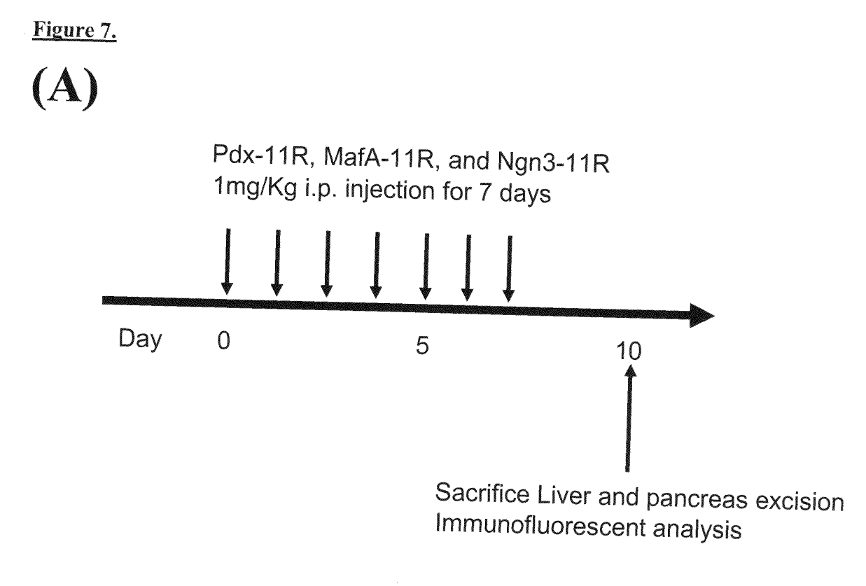

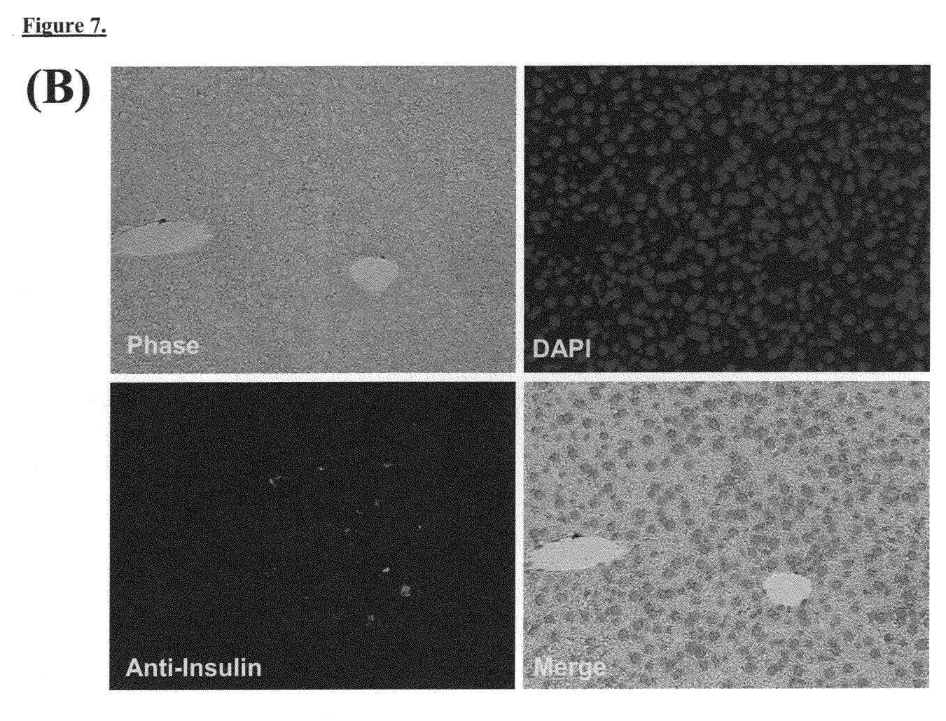

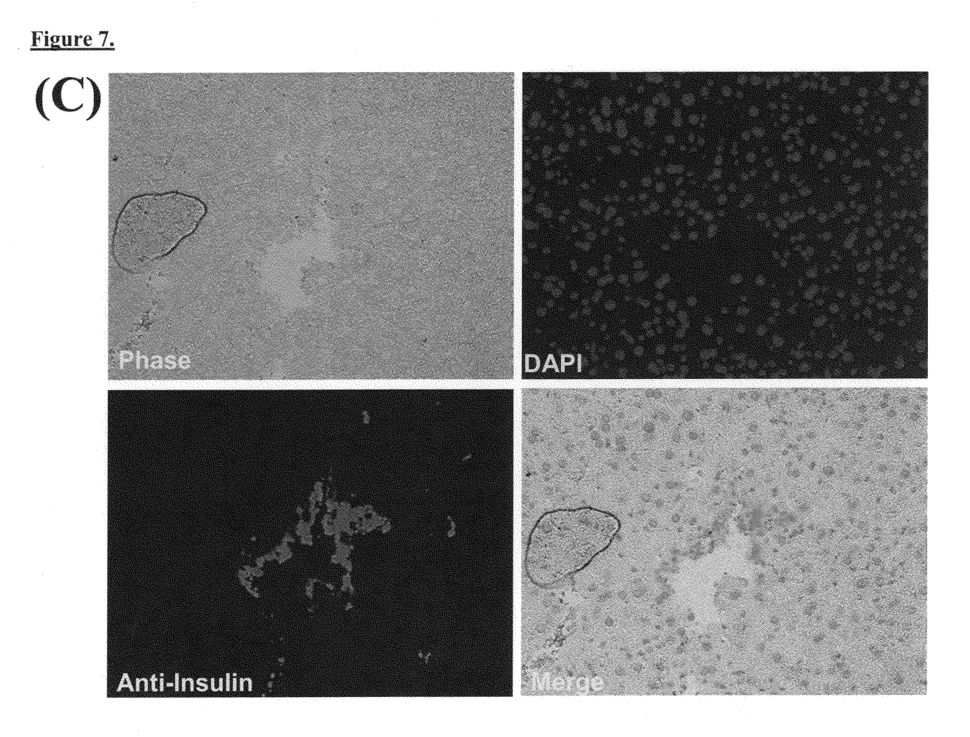

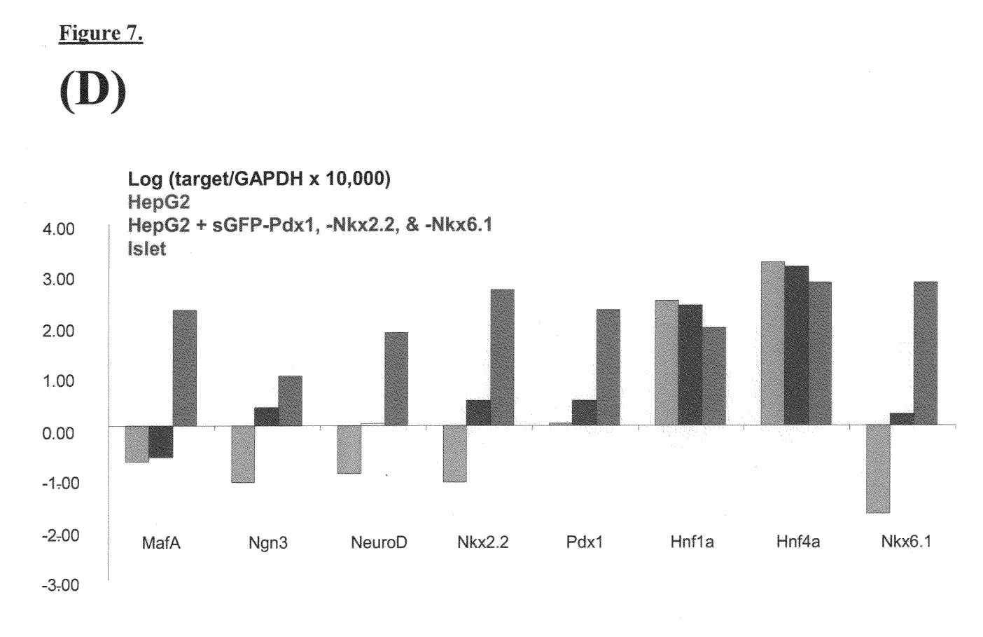

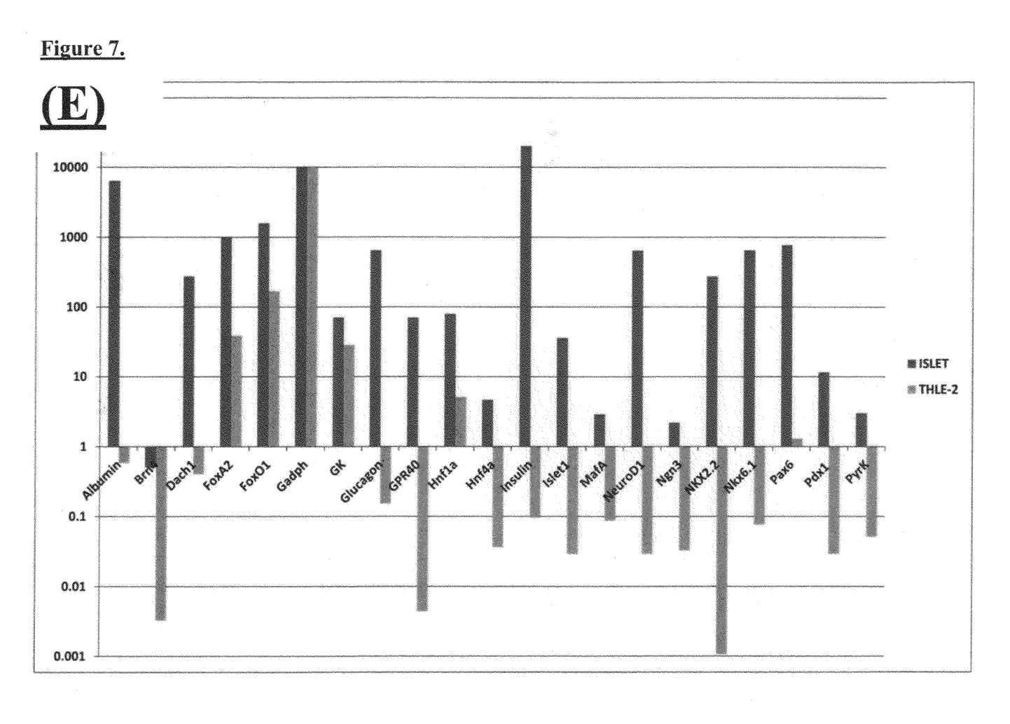

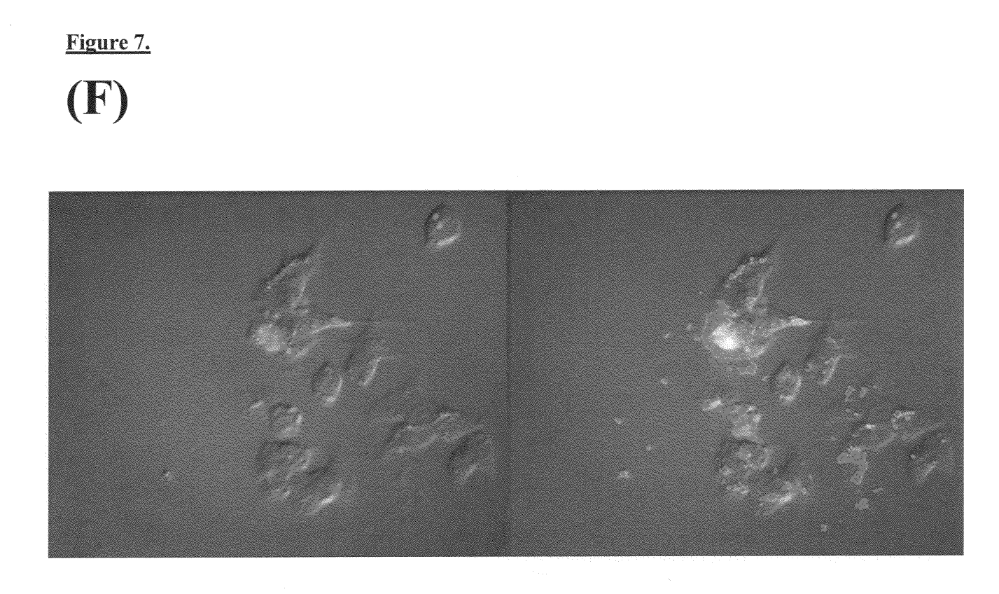

Following the experimental design of FIG. 7(A), wherein a combination of three transcription factors in the form of tagged proteins were injected (interperitoneal) into mice for 7 days, the triple protein treatment (Pdx1-11R, Ngn3-11R, and MafA-11R) produced insulin positive cells in mouse liver. A different Combination is also capable (sGFP-Pdx1, -Nkx2.2, and -Nkx6.1) shifted the gene expression profile of HepG2 cells towards that of islet cells as measured via qRT-PCR, verifying the network-based approach focusing on genetic regulatory architecture, as opposed to the particular capacity of a specific “morphogen” to direct differentiation.

### Example 3

### Conservation of β-Cell Regulatory Architecture

In human liver cell line demonstration of cell type transformation driven by selected exogenous transcription factors. As shown in FIG. 7(C), human liver cell lines have a very different genetic regulatory state compared to islet cells. Fluorescent microscopy shows that in FIG. 7(D) human liver cell line can be permanently transformed with a genetic expression construct containing the regulatory sequences that drive endogenous expression of the insulin gene (InsulinEnhancer-mCherry: red incorporated exogenous transcription factors: green). The insulin construct reports expression by transcription of a gene encoding the red fluorescent protein mCherry only in cells in which the regulatory state drives expression of the insulin gene. Human liver cells were treated three modified transcription factor proteins (Pdx1, Ngn3, and MafA−), which are expressed in pancreatic β cells identified as important regulators. The modified transcription factors were tagged with fluorescent green protein (GFP). These results show that most cells indeed incorporated these transcription factors with cells expressing mCherry under control of the insulin regulatory system (arrowhead). Given the wide divergence of original gene regulatory state, as shown by expression profile in 7(C), expression of the insulin construct unequivocally demonstrates that the exogenously applied transcription factors successfully induced a pancreatic regulatory state in some of the liver cells.

### Example 4

### Transformation of Non-Islet Cells of the Pancreas to Insulin-Secreting Cells

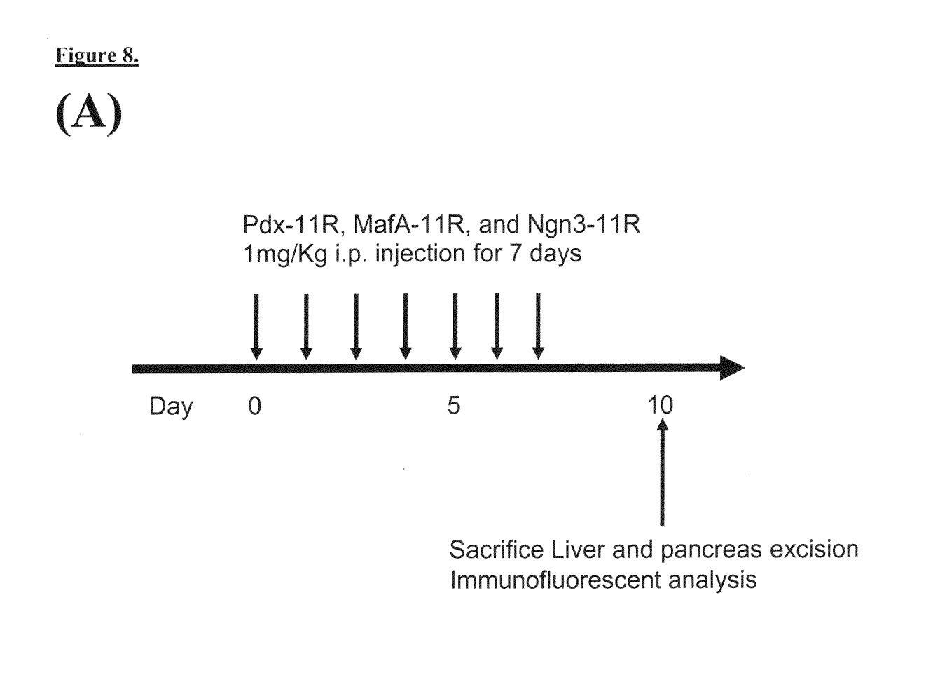

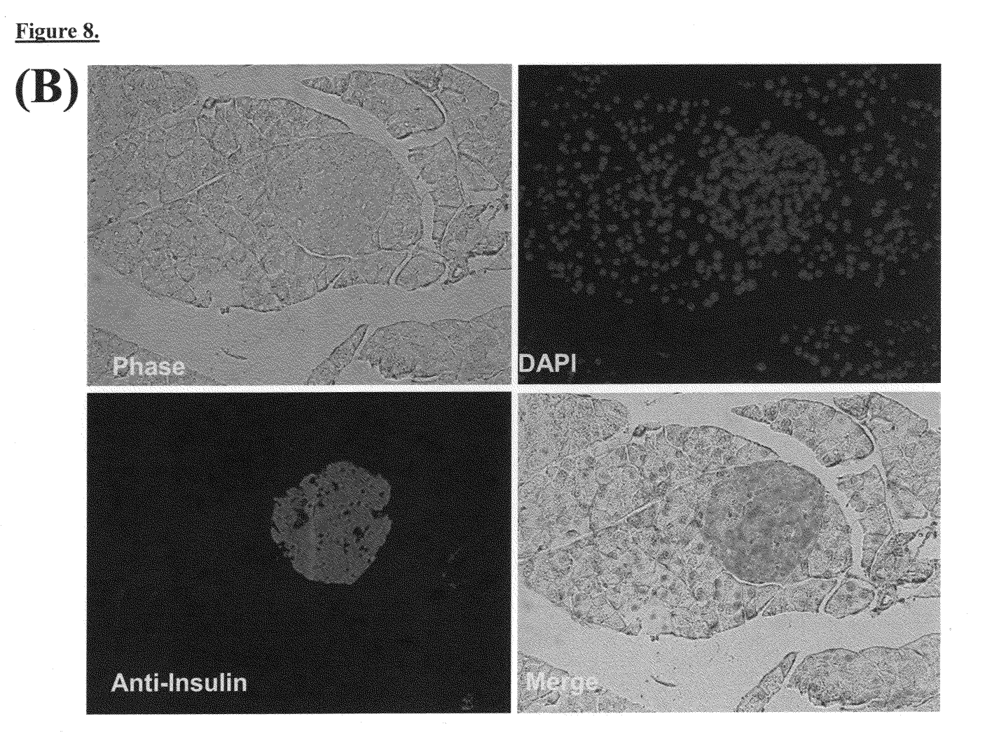

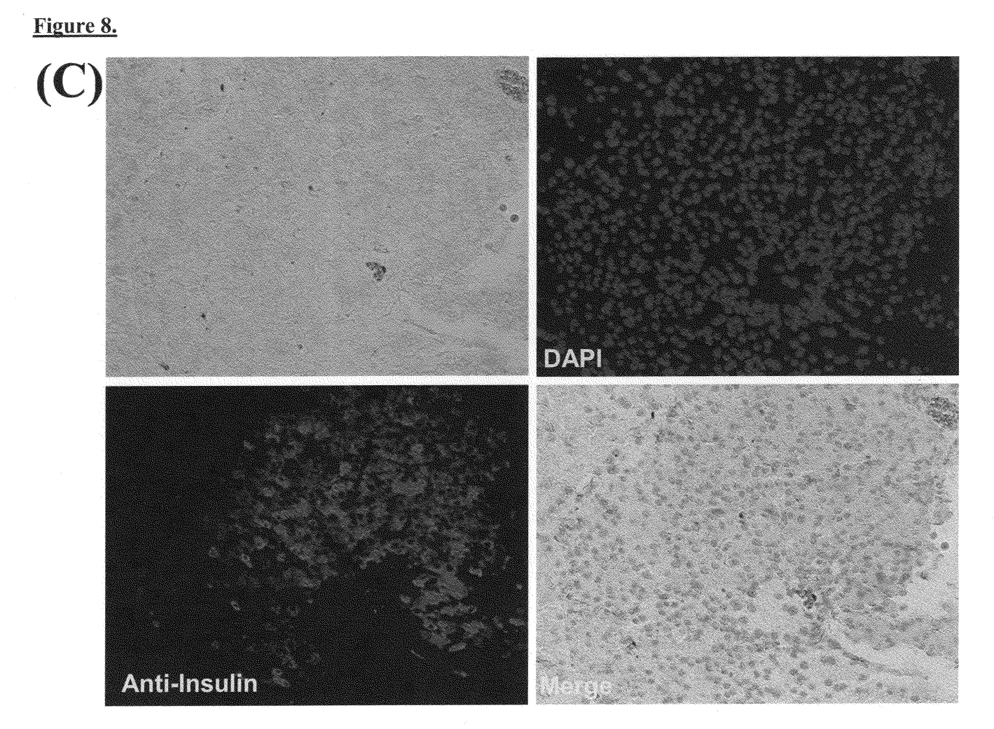

Using the experimental design in FIG. 8(A), successful transformation of non-islet cells of the pancreas to insulin-secreting cells is shown. Immunofluorescent analysis of control mouse pancreas showed that triple protein treatment (Pdx1-11R, Ngn3-11R, and MafA-11R) produced insulin positive cells in mouse pancreas FIG. 8 (B, C).

### Example 5

### Transformation of Peripheral T Cells to Treg Cells

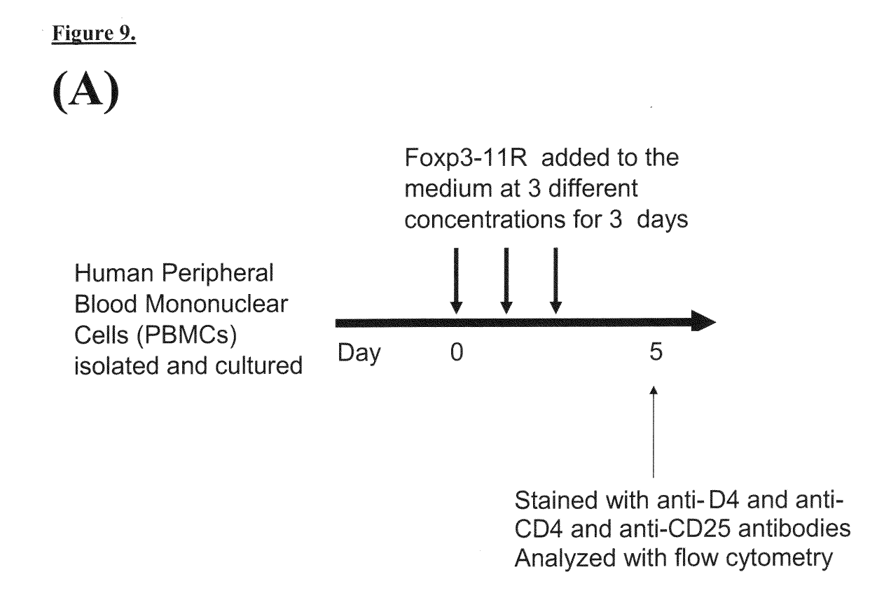

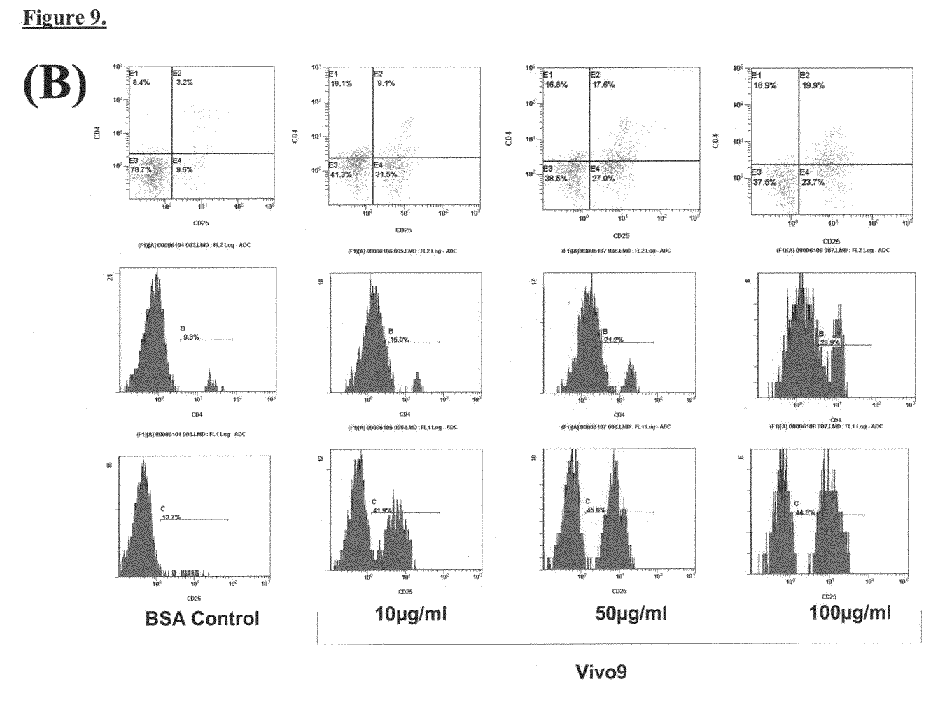

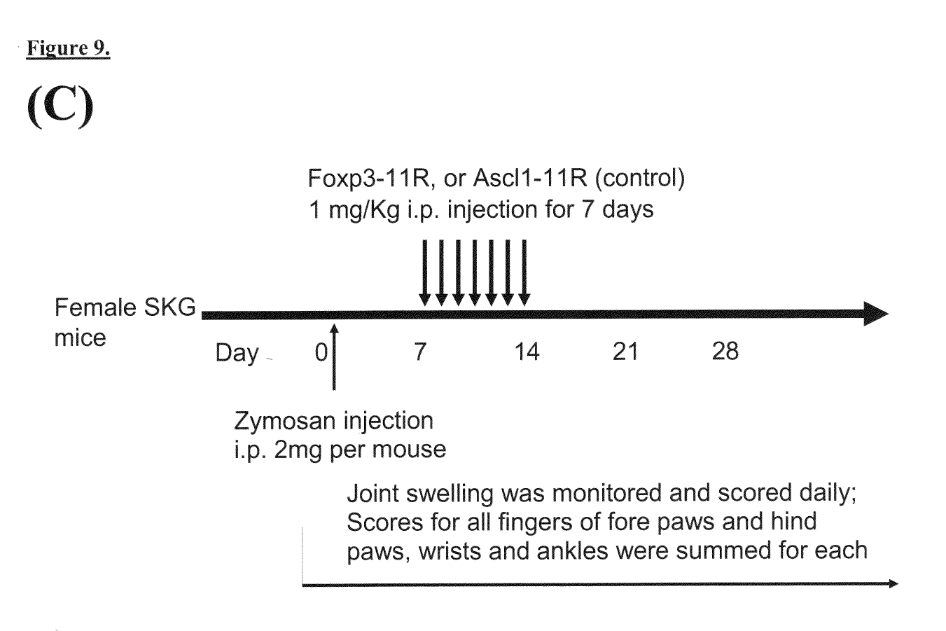

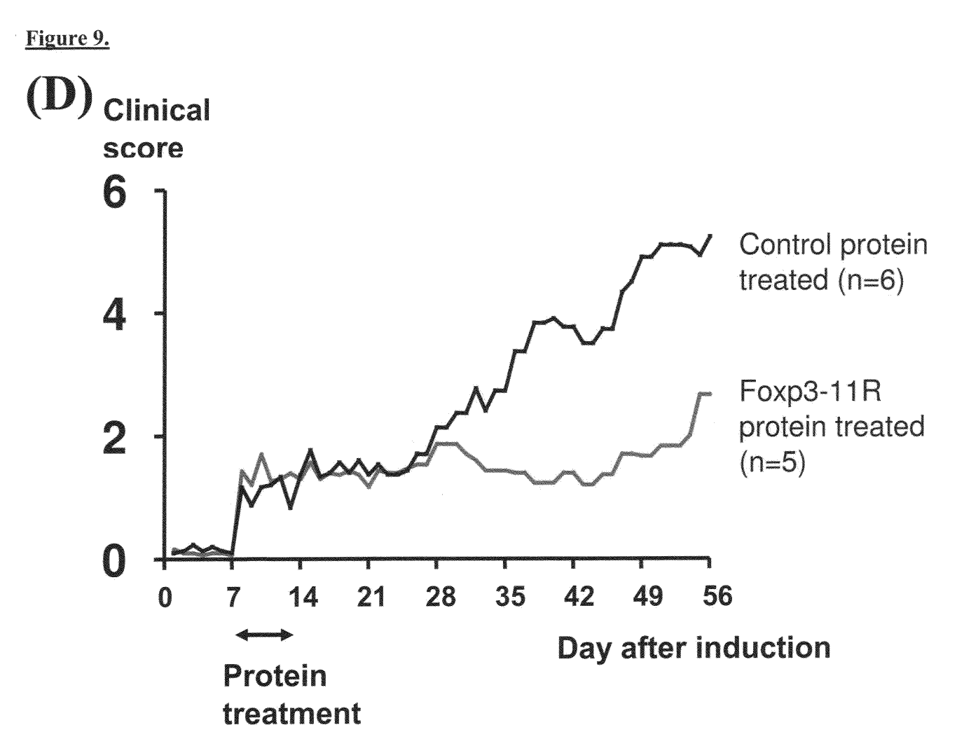

Using the experimental design in FIG. 9(A), successful transformation of peripheral T cells to Treg cells is shown. Application of Foxp3-11R increased the percentage of CD4+CD25Hi cells in a dose-dependent manner as shown via flow cytometry (FACS), FIG. 9(B). In addition, the application of transformed cell types for regenerative medicine application were demonstrated by application of deploying transformation in a disease model as shown in evaluation of Foxp3-11R in arthritis mouse model FIG. 9(C). The successful amelioration of rheumatoid arthritis in mouse model in FIG. 9(D) demonstrates in vivo cell reprogramming as capable of disease treatment.

### Example 6

### Transformation of Mesenchymal Stem Cells to Chondrocytes

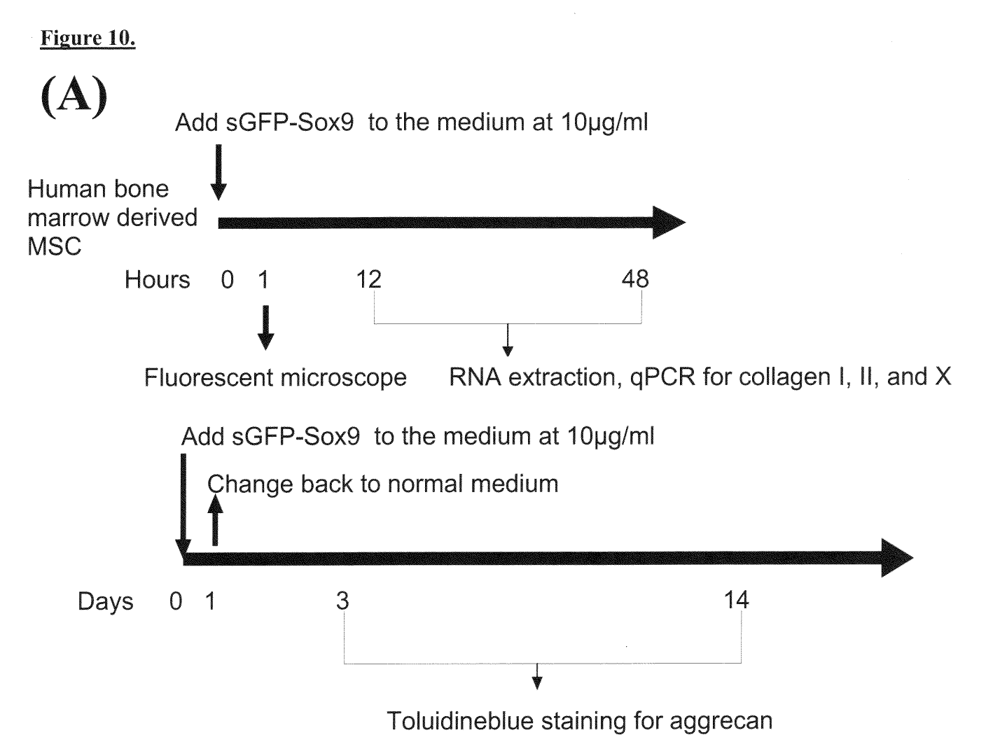

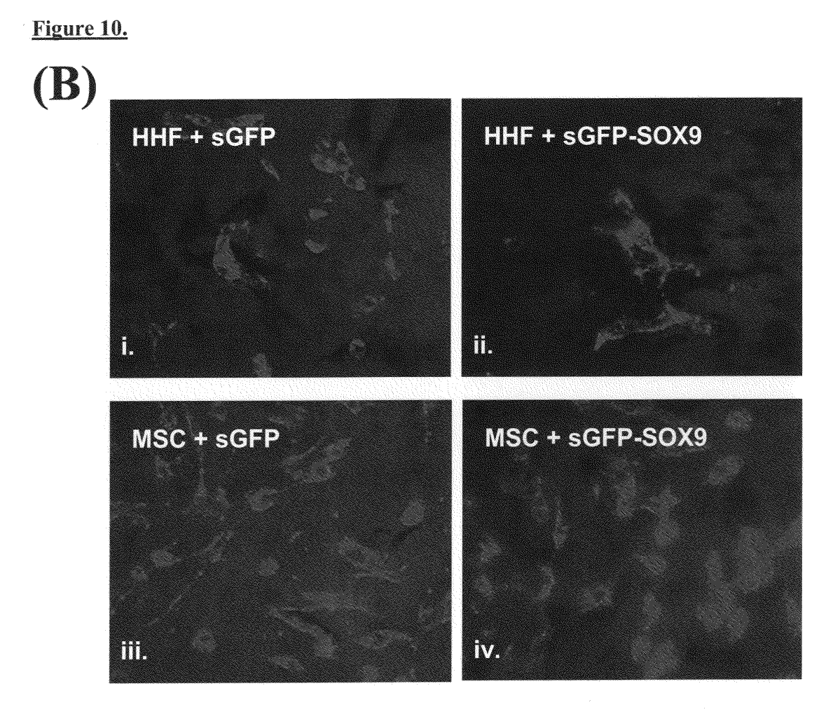

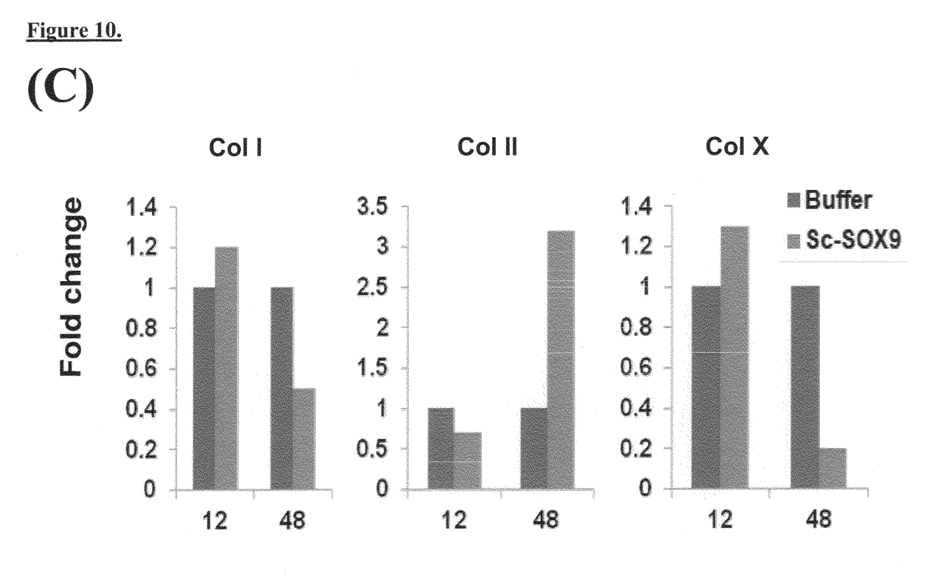

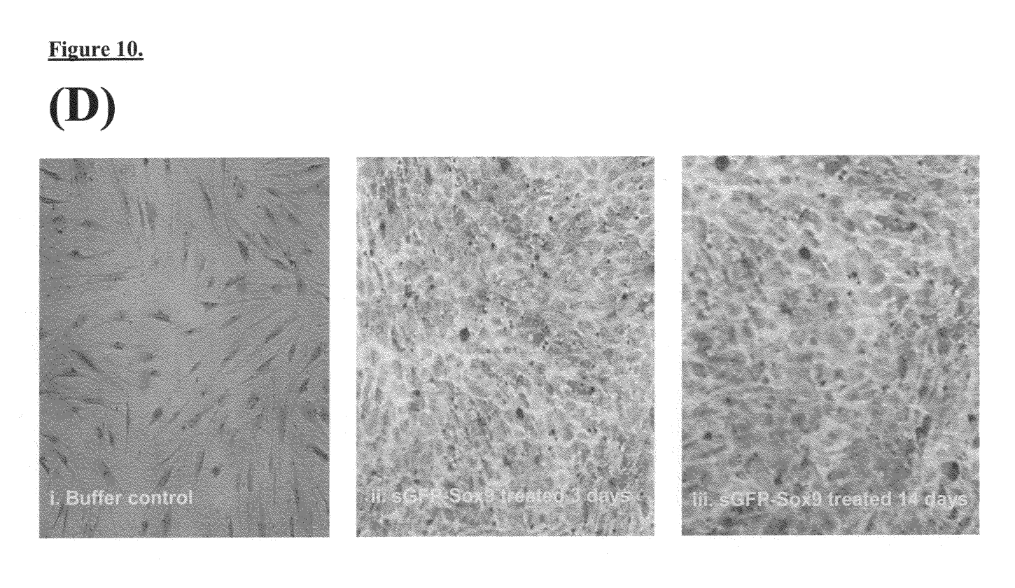

Using the experimental design in FIG. 10(A), successful transformation of mesenchymal stem cells to chondrocytes is demonstrated. It is first shown that a modified sGFP-SOX9 protein is capable of penetration into human skin fibroblast cell line, HHF, and human bone marrow derived mesenhymal stem cells, MSC FIG. 10(B) when incubated with 10 μg/ml of sGFP or sGFP-SOX9 in DMEM at 37° C. for 1 hour. Cells were washed and viewed under fluorescent microscope. i and iii: SGFP; ii and iv: sGFP-SOX9. Importantly, as shown in FIG. 10(C) sGFP-Sox9 increased collagen type II but decreased collagen type I and type X expression. MSC were cultured with DMEM with addition of buffer only or 10 μg/ml of sGFP-SOX9. At the indicated time point (hours), RNA were extracted and RT-PCR was performed with TagMan probe based analysis assay for collagen (Col) type I, II and X mRNA expression, as relative to GAPDH.

Extending the above results, it was further demonstrated, as depicted in FIG. 10(D) that sGFP-Sox9 increased aggrecan expression. 10 μg/ml of sGFP-SOX9 was added to MSC culture. After 24 hours, the MSCs were changed back to medium without sGFP-SOX9. Culture was maintained for 14 days. (i. MSC with buffer. ii. MSC with sGFPSOX9 treatment at 3 days. iii. MSC with sGFP-SOX9 treatment at 14 days. Toluidine blue staining) Toluidine blue stains aggrecan which is a major component of proteoglycan in articular cartilage matrix.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the methods transforming a cell, methods of modifying cells used in the described techniques, compositions of transformed cell generated by the aforementioned techniques, treatment of diseases and/or conditions that relate to the teachings of the invention, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of transforming a cell, comprising:
providing a quantity of at least one cis regulatory network element; and
introducing into a starting cell type, the at least one cis regulatory network element, wherein the at least one cis regulatory network element is capable of altering a regulatory sub-circuit in the starting cell type, thereby altering one of more properties of the starting cell type, and generating a transformed cell type.

2. The method of claim 1, wherein the cis regulatory network element comprises a transcription factor and derivatives thereof.

3. The method of claim 1, wherein the cis regulatory network element comprises a recombinant protein.

4. The method of claim 1, wherein the cis regulatory network element is encoded by a nucleic acid.

5. The method of claim 1, wherein the regulatory sub-circuit is a positive feedback loop.

6. The method of claim 5, wherein the regulatory sub-circuit comprises at least two cis regulatory network elements.

7. The method of claim 5, wherein the regulatory sub-circuit comprises at least three cis regulatory network elements.

8. The method of claim 1, wherein the one or more properties comprises transcription factor expression and/or transcription factor binding to a cis regulatory network element.

9. The method of claim 1, wherein the one or more properties comprises protein expression and/or surface marker expression.

10. The method of claim 1, wherein the starting cell type is a hepatocyte.

11. The method of claim 1, wherein the starting cell type is a non-insulin secreting islet cell.

12. The method of claim 1, wherein the transformed cell type is an insulin secreting islet cell.

13. The method of claim 12, wherein the insulin secreting islet cell expresses Pdx, MafA and Ngn3.

14. The method of claim 1, wherein the starting cell type is a peripheral T cell.

15. The method of claim 1, wherein the transformed cell type is a T_{reg} cell.

16. The method of claim 15, wherein the T_{reg} cell expresses Foxp3.

17. The method of claim 1, wherein the starting cell type is a mesenchymal stem cell.

18. The method of claim 1, wherein the transformed cell type is a chondrocyte.

19. The method of claim 18, wherein the chondrocyte expresses Sox9.

20. A quantity of transformed cells made by the method of claim 1.

21. A method for identifying a regulatory network for transforming a cell, comprising:
organizing a plurality of cis regulatory network elements into a network topology of nodes comprising circuits, wherein the circuits comprise at least one sub-circuit; and
identifying at least one sub-circuit comprising at least one positive effector node, wherein the at least one positive effector node is capable of generating a transformed cell type when introduced into a staring cell.

22. The method of claim 21, wherein the sub-circuit is a positive feedback loop.

23. The method of claim 22, wherein the sub-circuit comprises at least two cis regulatory network elements.

24. The method of claim 23, wherein the sub-circuit comprises at least three cis regulatory network elements.

25. A composition comprising:
a quantity of cells expressing at least one exogenously added protein, wherein the at least one exogenously added protein is a positive effector in a regulatory sub-circuit.

26. The composition of claim 25, wherein the cells express at least two exogenously added proteins, and the at least two exogenously added proteins are each positive effectors in a regulatory sub-circuit.

27. The method of claim 26, wherein the regulatory sub-circuit is a positive feedback loop.
